(19) United States
(12) Patent Application Publication
(10) Pub. No.: US 2022/0370535 A1
(43) Pub. Date: Nov. 24, 2022

(54) ANTIVIRAL ESSENTIAL OIL COMPOSITIONS

(71) Applicant: Hüseyin ERDOGMUS, Istanbul (KR)

(72) Inventors: Hüseyin ERDOGMUS, Stanbul (TR); Asli Deniz AYDIN, Stanbul (TR); Devrim Cagdas SON, Ankara (TR); Frank ALTINEL, Izmir (TR)

(21) Appl. No.: 17/594,708

(22) PCT Filed: Oct. 30, 2020
(86) PCT No.: PCT/TR2020/051021

§ 371 Date: Oct. 27, 2021

(51) Int. Cl. A61K 36/53; A61K 8/92; A61Q 17/00; A61K 45/06; A61L 9/03; A61L 9/013; A61Q 5/02; A61K 36/61; A61K 8/37; A61K 36/752; A61L 2/00; A61K 36/54; A61K 36/35; A61K 36/9068; C11B 9/00; A47K 10/32; A61K 36/83

(52) U.S. Cl. CPC A61K 36/53; A61K 8/922; A61Q 17/005; A61K 45/06; A61L 9/03; A61L 9/013; A61Q 5/02; A61K 36/61; A61K 8/37; A61K 36/752; A61L 2/0088; A61K 36/54; A61K 36/35; A61K 36/9068; C11B 9/0061; A47K 10/32; A61K 36/83; A47K 2010/3266

(57) ABSTRACT

Disclosed are antiviral essential oil compositions including one or more essential oils, such as styrax oil and resinoid, gurjun balsam oil, olibanum resinoid, nootka tree oil, vetiver oil, jatamansi oil, sandalwood oil, myrrhe oil, agarwood oil, copaiba balsam oil, ginger oil, curcuma oil, clary sage absolute, cypriol oil, rosemary oil, eucalyptus globulus, tea tree oil, patchouly oil, benzyl benzoate, vertofix(acetylated cedarwood oil), benzyl salicylate, benzyl cinnamate, cinnamyl benzoate, cinnamyl cinnamate, dihydroambrettolide, galaxolide, L-muscone, nootkatone, vetiveryl acetate, sclareol. The use of discovered fragrance molecules and essential oils alone or combination provides beneficial antiviral effects, especially for SARS-CoV-2 (COVID-19).

### TECHNICAL FIELD

The invention relates to antiviral essential oil compositions suitable for SARS-CoV-2 (COVID-19).

The invention especially relates to antiviral essential oil compositions comprising one or more essential oils and fragrance molecules, such as styrax oil and resinoid, gurjun balsam oil, olibanum resinoid, nootka tree oil, vetiver oil, jatamansi oil, sandalwood oil, myrrhe oil and resinoid, agarwood oil, copaiba balsam oil, ginger oil, curcuma oil, salvia sclaree oil, cypriol oil, rosemary oil, eucalyptus globulus, tea tree oil, patchouly oil, benzyl benzoate, vertofix(acetylated cedarwood oil), benzyl salicylate, benzyl cinnamate, cinnamyl benzoate, cinnamyl cinnamate, dihydroambrettolide, galaxolide, L-muscone, nootkatone, vetiveryl acetate, sclareol. The use of discovered fragrance molecules and essential oils, alone or combination provides beneficial antiviral effects, especially for SARS-CoV-2 (COVID-19).

### THE STATE OF THE ART

Coronaviruses (CoVs) belong to the family Coronaviridae which are enveloped, single-stranded, positive-sense RNA viruses. These viruses generally contain large (˜20 nm), surface projections called “spikes”, which in electron micrographs create an image reminiscent of the solar corona thus giving the name to the family. CoVs commonly cause respiratory problems but can also disrupt the digestive system or lead to systemic problems in mammals, birds and reptiles. In humans, they can cause very severe respiratory diseases such as SARS-CoV in 2002 and Middle East respiratory syndrome coronavirus (MERS-CoV) in 2012 [1]. The latest corona virus SARS-CoV-2, discovered in China, affected millions of people and killed hundreds of thousands worldwide. The World Health Organization (WHO) announced “COVID-19” as the name of this new disease caused by SARS-CoV-2 [2]. The ongoing SARS-CoV-2 threat that emerged in China has rapidly spread Worldwide and continuing to spread. Thus, many efforts have been directed to investigate drug(s) suitable for preventing and/or treating human SARS-CoV-2. Viruses affecting domesticated and wild animals pose significant economic and sustainability threats to commercial operations and natural ecosystems.

Viral infections of animals are being struggled by vaccines and pharmaceuticals that require evaluation and approval from health authorities. However, viral mutations can make many of these treatment methods ineffective. Moreover, insufficient resources and delayed regulatory approval can hamper vaccine and pharmaceutical development required to keep up with viral mutations. Vaccines and pharmaceuticals can also persist in the environment almost indefinitely and accumulate to biohazardous levels. Moreover, many antiviral treatments are inherently harmful to host subjects, but are used due to the lack of alternatives. Human immunodeficiency therapies are one example that is unable to completely eradicate HIV from the host person, but leaves the host subject's body and immune system in a compromised state.

As of the publication date of our study, there is no specific drugs or vaccines available to treat or prevent SARS-CoV-2. Several countries implemented drugs based on symptom-based therapies [3-5] to prevent further complications and organ damage [6]. There are various preliminary studies for treatment of SARS-CoV-2 infected patients. Anti-retroviral drugs such as remdesivir, lopinavir, ritonavir, oseltamivir is used in individual healing trials or animal experiments. From Wuhan Institute of Virology, Wang et. al. investigated some of the FDA(Food and Drug Administration) approved drugs and found that remdesivir and chloroquine could effectively inhibit the virus in cell-based assay with EC50 of 0.77 and 1.13 μM, respectively [7]. Similar studies reported that, combination of protease inhibitor lopinavir/ritonavir could be used for treatment of SARS-CoV-2.

Other antiviral treatments include nucleoside analogues, neuraminidase inhibitors, umifenovir (arbidol), tenofovir disoproxil (TDF), and lamivudine (3TC) [8]. According to binding free energy calculations using the molecular mechanics, Xu et al. indicated that among 4 tested drugs (nelfinavir, pitavastatin, perampanel, and praziquantel) nelfinavir was identified as the most potential inhibitor against SARS-CoV-2, Mpro [9]. In addition, alternative traditional Chinese medicine implementations have been reported [3, 4, 10, 11]. Although, results from these preliminary studies remain unapproved for therapeutic use in clinical settings they are still very valuable for drug studies against the current pandemic. In order to speed up possible clinical trials and drug discovery against SARS-CoV-2, many compounds that are being used as drugs or supplements for humans are started to be tested as potential lead molecules. Molecules that are going to be implemented as antiviral treatment and protection have several requirements: First of all, stock of the drug must be sufficient and readily available; the safety of treatment should be tolerated by the patients and finally the cost should be as low as possible.

A patent application document numbered US2020237689 A1 relates to nanoemulsion compositions with certain surfactant blend ratios that impart enhanced permeability. The document states that the compositions are suitable for mucosal and intranasal applications and allow for the greater delivery of one or more active agents to the application site to prevent infection by coronavirus.

### PURPOSE OF THE INVENTION

The present invention relates to antiviral essential oil compositions meeting the needs mentioned above, eliminating all disadvantages, and providing some additional advantages.

Main purpose of the invention is to provide new compositions comprising essential oils and fragrance molecules alone or combination for antiviral effects, especially for SARS-CoV-2 (COVID-19).

Another purpose of the invention is to prepare—not limited to these formulations—hand sanitizer, anti-bacterial/anti-viral cologne, shampoo, electrical diffuser formulation, reed diffuser formulation, air freshener & sanitizer formulation, air nebulizer formulation, hand cream formulation, wet wipe formulation, cleaner formulation to prevent and/or cure SARS-CoV-2 infection.

Another purpose of the invention is to use easily obtainable, renewable, inexpensive, all-natural herbal essential oils for obtaining antiviral compositions. These essential oils have been used for many years and are extremely safe. Their application can be used like a cosmetic and perfumery product without requiring any medical and technical skills.

In order to achieve above mentioned purposes, the invention is an antiviral composition to prevent and/or cure SARS-CoV-2 infection. The antiviral composition of the invention can comprise Formula A, Formula B, Formula C, Formula D, Formula E, Formula F and Formula G alone or combination.

The structural and characteristics features of the invention and all advantages will be understood better in detailed descriptions below, and therefore, the assessment should be made taking into account the said detailed explanations.

### BRIEF DESCRIPTION OF THE DRAWINGS

FIG. 1—AutoDock binding pose of 2-Methoxyfuranadiene using BRD2 as the target protein. Amino acids in the interaction distance to the ligand were labeled in the dock poses.

FIG. 2—AutoDock binding pose of Beta vetivone using BRD2 as the target protein. Amino acids in the interaction distance to the ligand were labeled in the dock poses.

FIG. 3—AutoDock binding pose of Cinnamyl cinnamate using BRD2 as the target protein. Amino acids in the interaction distance to the ligand were labeled in the dock poses.

FIG. 4—AutoDock binding pose of Curzerene using BRD2 as the target protein. Amino acids in the interaction distance to the ligand were labeled in the dock poses.

FIG. 5—AutoDock binding pose of Dihydro Karanone using BRD2 as the target protein. Amino acids in the interaction distance to the ligand were labeled in the dock poses.

FIG. 6—AutoDock binding pose of Incensol using BRD2 as the target protein. Amino acids in the interaction distance to the ligand were labeled in the dock poses.

FIG. 7—AutoDock binding pose of Isopropyl trimethylcyclotetradecatrienol using BRD2 as the target protein. Amino acids in the interaction distance to the ligand were labeled in the dock poses.

FIG. 8—AutoDock binding pose of Jinkoheremol using BRD2 as the target protein. Amino acids in the interaction distance to the ligand were labeled in the dock poses.

FIG. 9—AutoDock binding pose of Karanone using BRD2 as the target protein. Amino acids in the interaction distance to the ligand were labeled in the dock poses.

FIG. 10—AutoDock binding pose of Kusunol(valerianol) using BRD2 as the target protein. Amino acids in the interaction distance to the ligand were labeled in the dock poses.

FIG. 11—AutoDock binding pose of Lindestrene using BRD2 as the target protein. Amino acids in the interaction distance to the ligand were labeled in the dock poses.

FIG. 12—AutoDock binding pose of Muskaton using BRD2 as the target protein. Amino acids in the interaction distance to the ligand were labeled in the dock poses.

FIG. 13—AutoDock binding pose of Nootkatone using BRD2 as the target protein. Amino acids in the interaction distance to the ligand were labeled in the dock poses.

FIG. 14—AutoDock binding pose of Sclareol using BRD2 as the target protein. Amino acids in the interaction distance to the ligand were labeled in the dock poses.

FIG. 15—AutoDock binding pose of Vetiveryl acetate using BRD2 as the target protein. Amino acids in the interaction distance to the ligand were labeled in the dock poses.

FIG. 16—AutoDock binding pose of 2-Methoxyfuranadiene using MPro as the target protein. Amino acids in the interaction distance to the ligand were labeled in the dock poses.

FIG. 17—AutoDock binding pose of Beta vetivone using MPro as the target protein. Amino acids in the interaction distance to the ligand were labeled in the dock poses.

FIG. 18—AutoDock binding pose of Cinnamyl Cinnamate using MPro as the target protein. Amino acids in the interaction distance to the ligand were labeled in the dock poses.

FIG. 19—AutoDock binding pose of Curzerene using MPro as the target protein. Amino acids in the interaction distance to the ligand were labeled in the dock poses.

FIG. 20—AutoDock binding pose of Dihydro Karanone using MPro as the target protein. Amino acids in the interaction distance to the ligand were labeled in the dock poses.

FIG. 21—AutoDock binding pose of Incensol using MPro as the target protein. Amino acids in the interaction distance to the ligand were labeled in the dock poses.

FIG. 22—AutoDock binding pose of Isopropyl trimethylcyclotetradecatrienol using MPro as the target protein. Amino acids in the interaction distance to the ligand were labeled in the dock poses.

FIG. 23—AutoDock binding pose of Jinkoheremol using MPro as the target protein. Amino acids in the interaction distance to the ligand were labeled in the dock poses.

FIG. 24—AutoDock binding pose of Karanone using MPro as the target protein. Amino acids in the interaction distance to the ligand were labeled in the dock poses.

FIG. 25—AutoDock binding pose of Kusunol(valerianol) using MPro as the target protein. Amino acids in the interaction distance to the ligand were labeled in the dock poses.

FIG. 26—AutoDock binding pose of Lindestrene using MPro as the target protein. Amino acids in the interaction distance to the ligand were labeled in the dock poses.

FIG. 27—AutoDock binding pose of Muskaton using MPro as the target protein. Amino acids in the interaction distance to the ligand were labeled in the dock poses.

FIG. 28—AutoDock binding pose of Nootkatone using MPro as the target protein. Amino acids in the interaction distance to the ligand were labeled in the dock poses.

FIG. 29—AutoDock binding pose of Sclareol using MPro as the target protein. Amino acids in the interaction distance to the ligand were labeled in the dock poses.

FIG. 30—AutoDock binding pose of Vetiveryl acetate using MPro as the target protein. Amino acids in the interaction distance to the ligand were labeled in the dock poses.

FIG. 31—LigPlot+ diagrams of protein-ligand interactions for Sclareol, Benzyl Benzoate, Benzyl Salicylate using main protease as the target protein. Amino acids that can generate hydrogen bonds with the ligand were printed in bold. An arc represents hydrophobic contacts with spokes radiating towards the ligand atoms they contact.

FIG. 32—LigPlot+ diagrams of protein-ligand interactions for Sclareol, Benzyl Benzoate, Benzyl Salicylate using BRD2 as the target protein. Amino acids that can generate hydrogen bonds with the ligand were printed in bold. An arc represents hydrophobic contacts with spokes radiating towards the ligand atoms they contact.

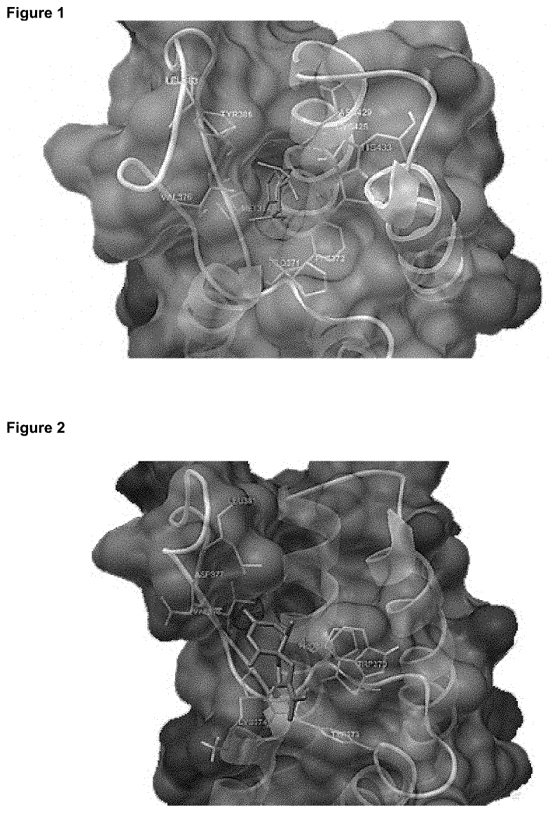

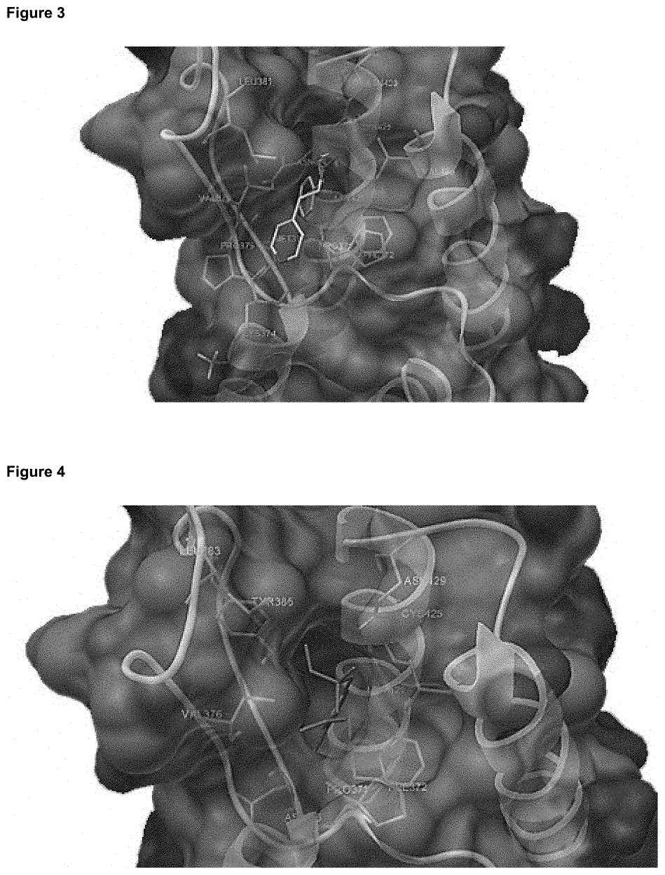

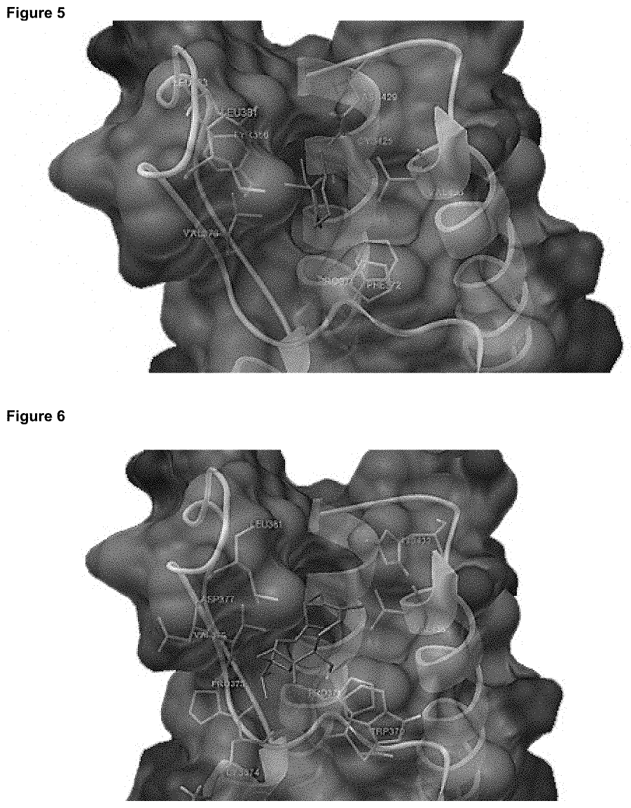

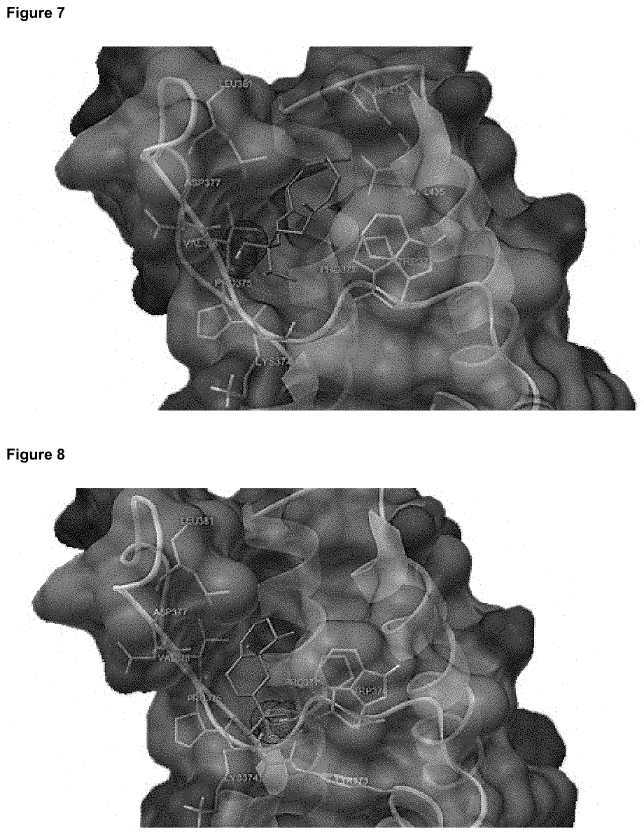

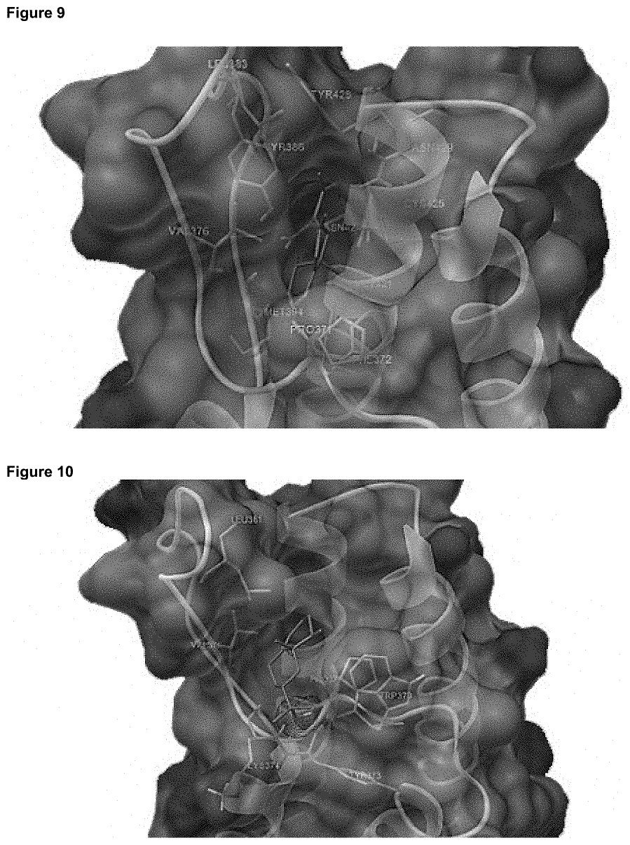

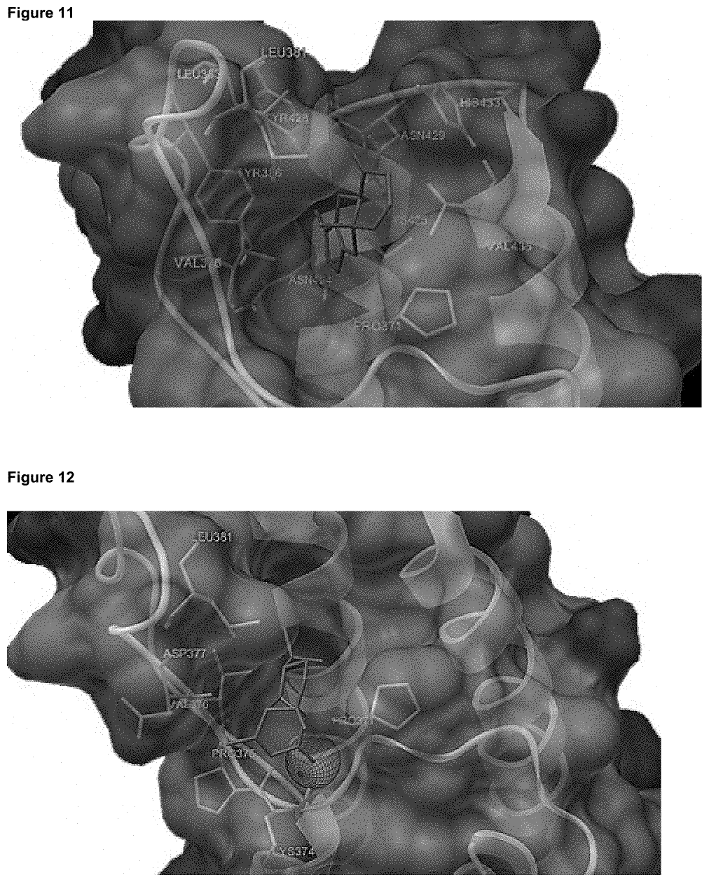

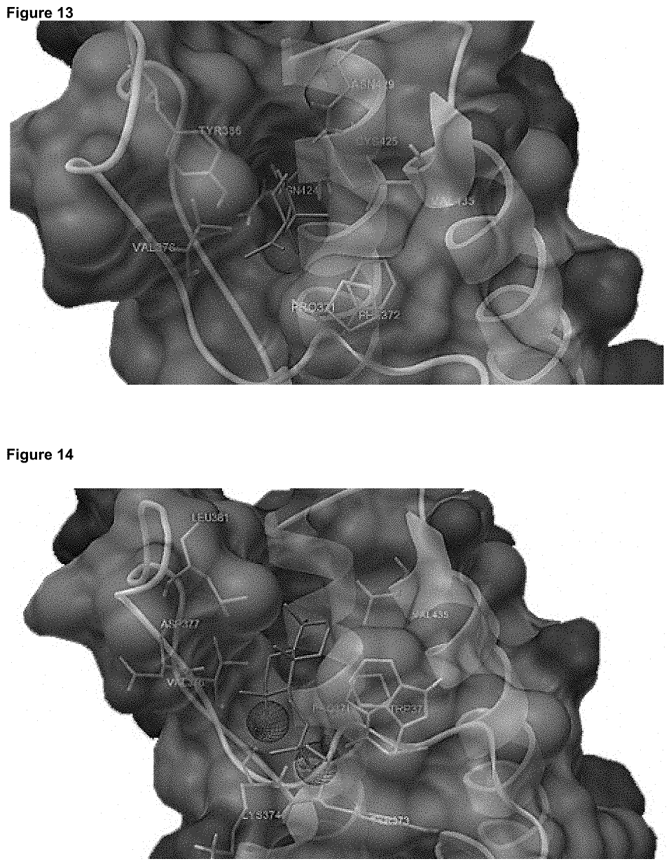

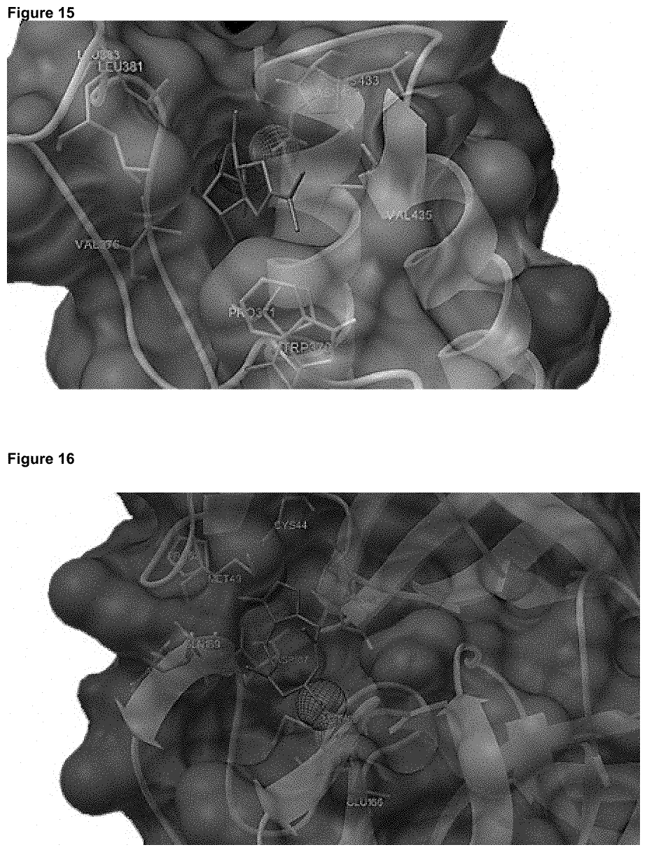

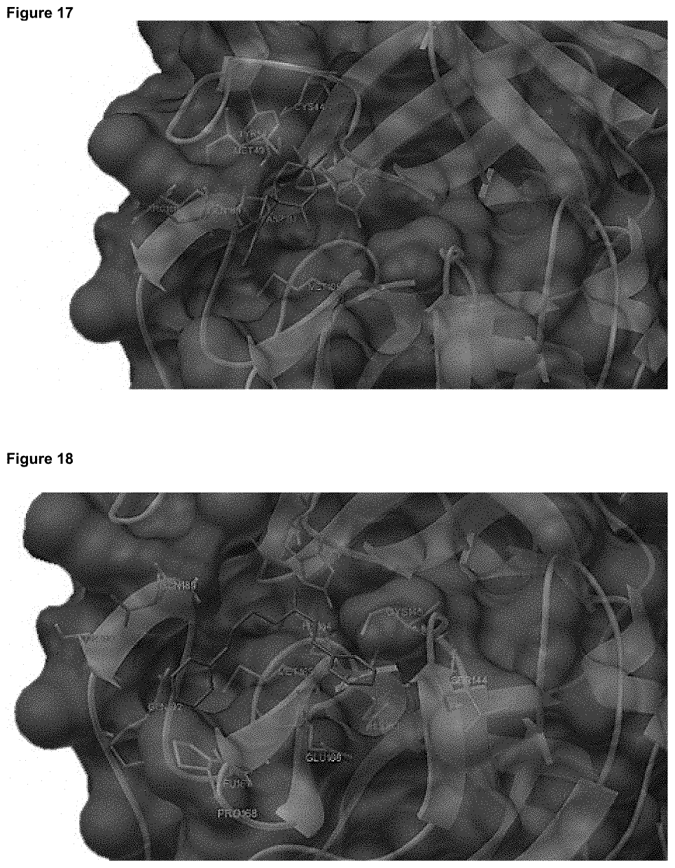

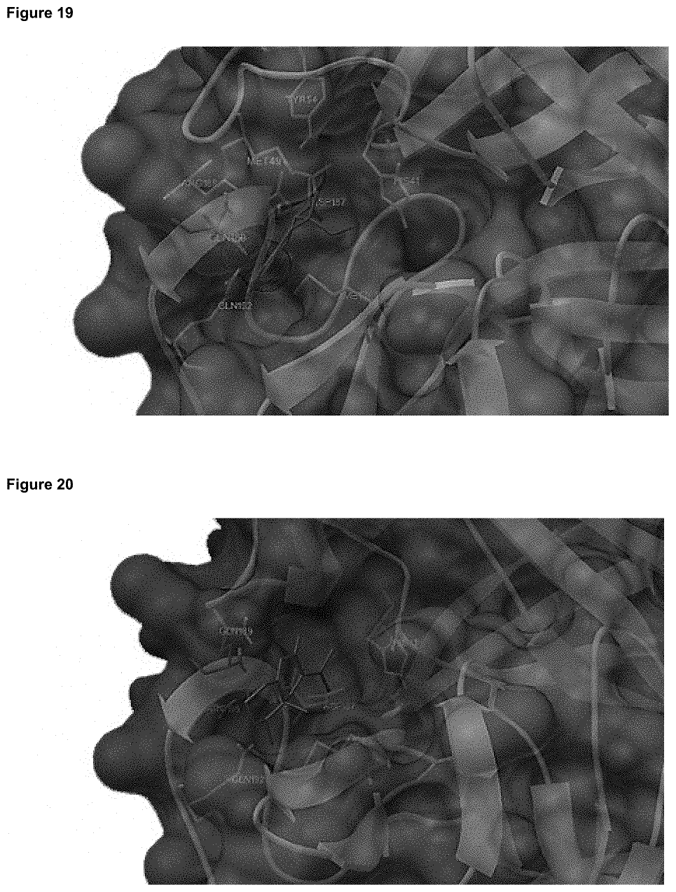

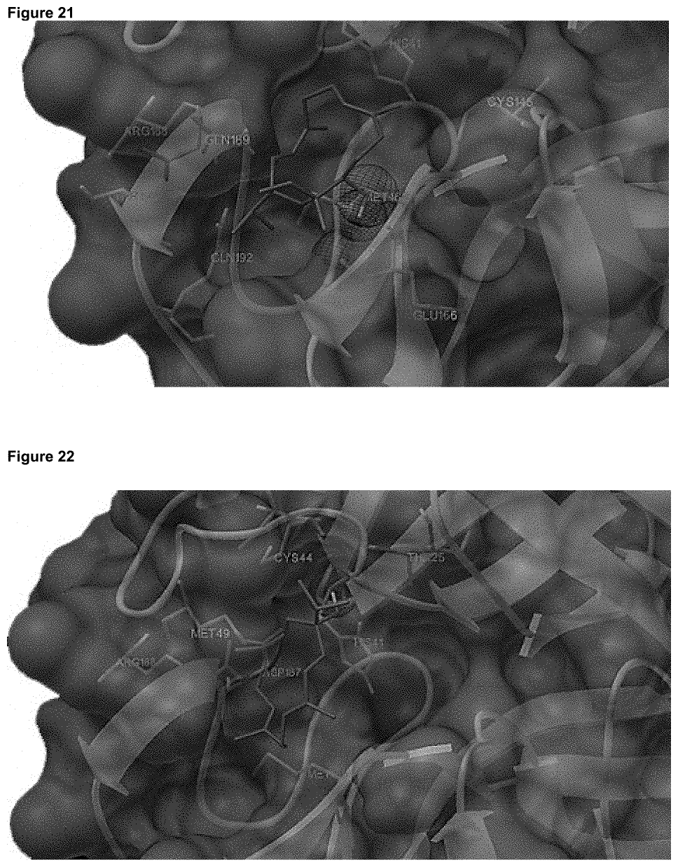

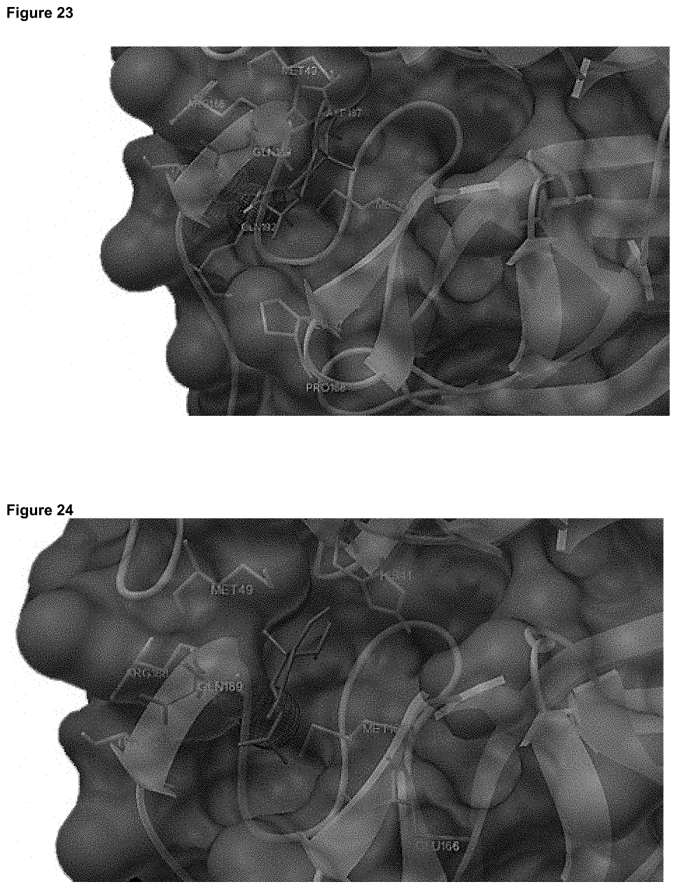

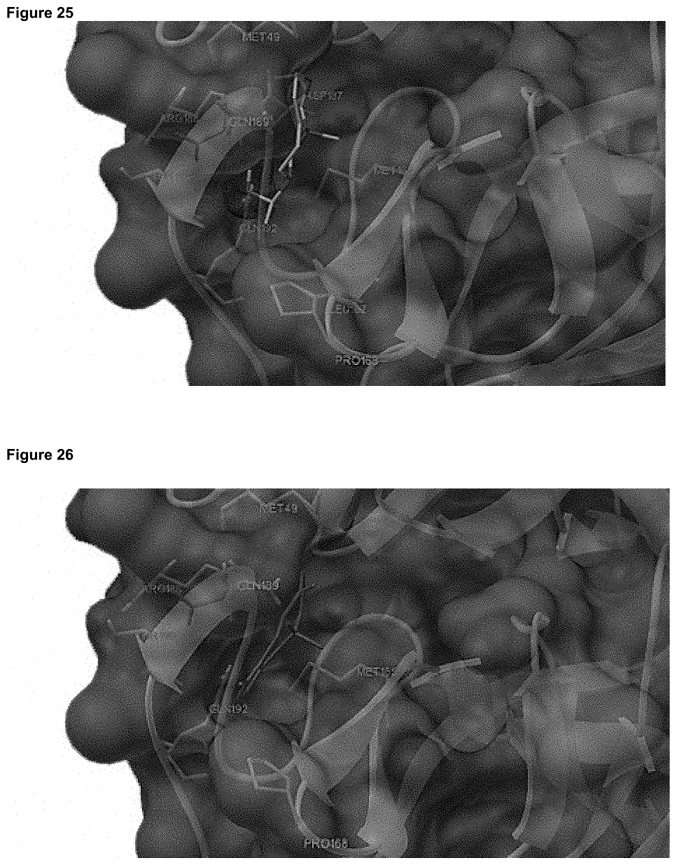

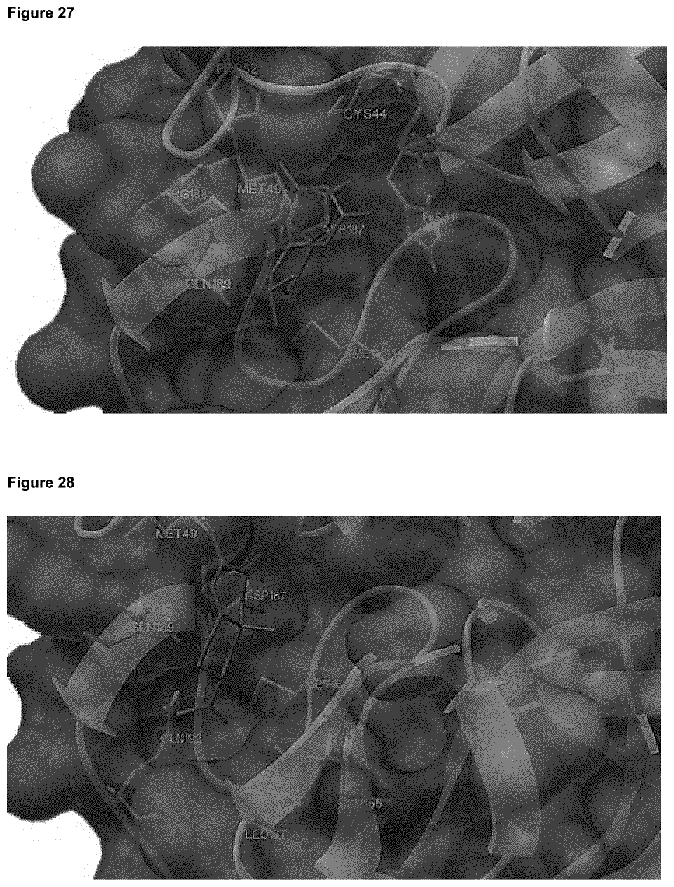

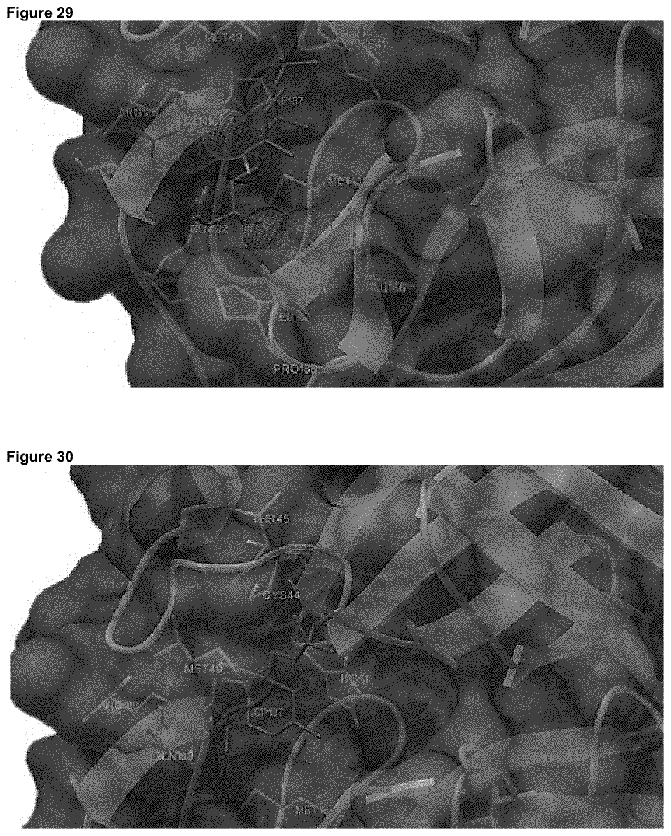

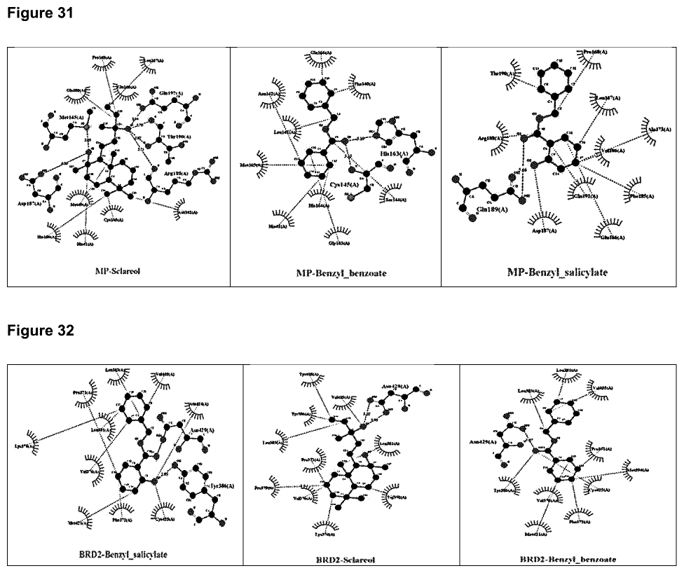

### DETAILED DESCRIPTION OF THE INVENTION

In this detailed description, antiviral essential oil composition has been described in a manner not forming any restrictive effect and only for purpose of better understanding of the matter.

The invention relates to antiviral essential oil compositions including one or more essential oils and molecules, such as styrax oil and resinoid, gurjun balsam oil, olibanum resinoid, nootka tree oil, vetiver oil, jatamansi oil, sandalwood oil, myrrhe oil and resinoid, agarwood oil, copaiba balsam oil, ginger oil, curcuma oil, clary sage absolute, cypriol oil, rosemary oil, eucalyptus globulus, tea tree oil, patchouly oil, 2-methoxyfuranodiene, 2-acetoxyfuranodiene, 2-hydroxyfuranodiene, lindestrene, curzerene, beta caryophyllene, rotundone, alpha bulnesene, alpha guaiene, agorospirol, alpha agarofuran, dihydro karanone, karanone, jinkoheremol, gamma gurjunene, beta gurjunene, alpha gurjunene, aromadendrene, delta cadinene, delta elemene, beta elemene, alpha cubebene, alpha ylangene, incensol and isopropyl trimethylcyclotetradecatrienol, alpha cyperone, cyperene, cyprotundone, phenylpropyl cinnamate, cinnamyl cinnamate, benzyl cinnamate, cinnamyl benzoate, benzyl benzoate, benzyl salicylate, alpha copaene, khusimon, beta vetivone, alpha zingiberene, beta selinene, beta sesquiphellendrene, beta bisabolene, alpha bisabolene, alpha curcumene, alpha farnesene, vetiveryl acetate, khusimon, beta-vetivone, L-Muscone, alpha bergamotenol, cis-nuciferol, cis-lanceol, alpha bisabolol, Jatamansone, valerianol, valerenal, alpha curcumene, Galaxolide, dihydroambrettolide, vertofix (acetylated alpha cedrene and thujopsene), nootkatone, sclareol. The use of discovered fragrance molecules and essential oils, alone or combination provides beneficial antiviral effects, especially for SARS-CoV-2 (COVID-19).

An essential oil is a volatile material, derived by a physical process from odorous plant material of a single botanical form and species.

Essential oils generally constitute the odorous principles of the plants in which they exist.

The essential oils are either distilled, expressed or extracted. Expression is performed exclusively in the cases of peels of citrus fruits. Extracts are prepared materials.

The term extract is used for perfume materials, flavour materials, pharmaceutical products and many other commercial products. Absolutes, Concretes, Extraits (dissolved extracts), Oleoresins (prepared), Resinoids, and Tinctures are forms of Extracts.

Essential Oils are complex natural mixtures of aromatic secondary metabolites, extracted from herbs, peels, flowers, fruits, seeds, leaves barks, woods, roots, lichens, resins, gums and secretions. The chemical compounds of Essential oils, mainly monoterpenes, sesquiterpenes, oxygenated terpenes, oxygenated sesquiterpenes and phenylpropanoids. Hydrocarbons, alcohols, esters, aldehydes, phenols, acids, ethers and ketones are responsible for the fragrance and pharmacological activities.

Most Essential oils and their characteristic compounds have several biological properties such as anti-stress, anti-aging, antibacterial, antifungal, antiviral, antioxidant, anti-inflammatory, wound healing and anti-cancer effects.

Throughout the evolutionary history of life on Earth, essential oils in plants and trees have been theorized to evolve with viruses, bacteria and fungi to protect plants and trees from viral, bacterial and fungal infections. Therefore, by looking at the anti-viral behaviour of the plants, it can be easily seen that the defence mechanism is based on essential oils. In this context, different fragrance ingredient categories for anti-viral activities were reviewed. Virucidal activity of essential oils, which are lipophilic by nature, is probably due to disruption of the viral membrane or interference with viral envelope proteins involved in host cell attachment.

Considering the potential antiviral effects of these natural molecules docking simulations are carried out in order to identify drug target molecules against SARS-CoV-2. For this purpose, two functional proteins that has structure information submitted to Research Collaboratory for Structural Bioinformatics Protein Data Bank (RCSB-PDB) and reported as essential for SARS-CoV-2 spread were picked. These proteins include the SARS-CoV-2, Main Protase (Mpro) and Host Bromodomain protein (BRD2).

First protein that is used in this study is the target protein used in many recent studies, the Main protease which is also named as chymotrypsin-like protease. This protease can cleave many sites in the polyproteins and generate non-structural proteins (nsps) that play role in the assembly of replicase-transcriptase complex (RTC). Inhibition of this protease suggested as a potential drug that can prevent the spread of SARS-CoV-2. The crystal structure of this protein was resolved by several groups and RCSB-PDB contains many entries for this protein. In studies, the structure of SARS-CoV-2 main protease bound to potent broad-spectrum non-covalent inhibitor X77 (6W63) [18] is used.

The second protein in our study is a host protein which is a member of the bromodomain extra terminal protein family. This family proteins known to regulate expression of —1,450 genes. Early reports from the cell mapping initiative at Quantitative Biosciences Institute Coronavirus Research Group indicate that BRD2 a member of bromodomain protein family may interact with SARS-CoV-2 envelope proteins. The bromodomain proteins recognize and bind to acetylated histones and play a critical role for host's hype-immune response. SARS-CoV-2 virus protein E mimics acetylated histones and could bind to the same site on BRD-2 [19]. It has been suggested that BRD2 inhibitors can potentially block where SARS-CoV-2 envelop protein E binds and could be used as drug targets. In this study, the used crystal structure (5UEW) of BRD2 is obtained from RCSB-PDB [20].

Analysis of essentials oils for identifying the molecules were performed by GC/MS (Gas chromatography coupled with Mass Spectrometry. The instruments used are Agilent 6890 gas chromatograph equipped with a split/splitless inlet in combination with Agilent 5973N MSD. Separations were done 50 m×0.32 mm id Carbowax 20 M and SE 54 columns. The oven heated 3° C. per minute from 60° C. to 260° C. and hold for five minutes at 260° C. in both polar and non-polar columns. Separated components are identified by in-house EPS Fragrances MS library, Wiley Registry 10th Edition/Nist 2014 Mass Spectrum Library (W10n14), NIST 2017 Mass Spectral Library (Nist17), Adams Essential oil Identification Mass Spectrum Library (Adams) [21-26].

Analysed Essential Oils to Identify the Molecules

Agarwood Oils (Aquilaria Malacsensis, Aquilaria Crassna, Aquilaria Sinensis)

Ambergris (Ambra Grisea)

Ambrette Seed Oil (Hibiscus Abelmoschus)

Amyriss Oil (Amyris Balsamifera)

Angelica Root Oil (Angelica Archangelica)

Angelica Seed Oil (Angelica Atchangelica)

Anise Oil (Pimpinella Anisum)

Araucaria Oil (Callitropsis Araucarioldes)

Arnica Oil (Arnica Montana)

Artemisia Oil (Artemisia Vulgaris)

Sweet Wormwood Oil (Artemisia Annua)

Assafoedia Oil (Ferrula Assafoetida)

Fir Balsam Absolute (Abies Balsamea)

Basil Oil (Ocimum Basilicum)

Bay Leaf Oil (Pimenta Racemosa)

Benzoin Resinoid Siam (Styrax Tonkinensis)

Bergamot Oil (Citrus Bergamia)

Bette! Oil (Piper Betle)

Black Cumin Oil (Nigella Sativa)

Black Currant Absolute (Ribes Nigrum)

Black Pepper Oil (Piper Nigrum)

Boronia Absolute (Boronia Megastigma)

Broom Absolute (Spartium Junceum)

Buchu Leaf Oil (Buchu Leaf Oil)

Cabreuva Oil (Myrocarpus Fastigiatus)

Cajuput Oil (Melaleuca Leucadendron)

Calamus Oil (Acorus Calamus)

Camphor Oil (Cinnamomum Camphora)

Cananga Oil (Cananga Odorata)

Caraway Oil (Carum Carvi)

Cardamom Oil (Elettaria Cardamomum)

Carrot Seed Oil (Daucus Carota)

Cassia Oil (Cinnamomum Cassia)

Cassie Absolute (Acacia Farnesiana)

Castoreum Absolute (Castor Canadensis)

Cedarleaf Oil (Thuja Occidentalis)

Cedarwood Atlas Oil (Cedrus Atlantica)

Cedarwood Virginia Oil (Juniperus Virginiana)

Celery Seed Oil (Apium Graveolens)

Chamomile Oil Blue (Matricaria Chamomilla)

Roman Chamomile Oil (Anthemis Nobilis)

Champaca Absolute (Michelia Champaca)

Cinnamon Bark Oil (Cinnamomum Zeylanicum)

Cistus Oil (Cistus Ladaniferus)

Cistus Absolute (Cistus Ladaniferus)

Citronella Oil (Cymbopogon Nardus)

Civet Absolute (Viverra Zibeth)

Clove Bud Oil (Eugenia Caryophyllata)

Copaiba Balsam (Copaifera Reticulata)

Coriander Oil (Coriandrum Sativum)

Costus Oil (Saussurea Lappa)

Cubeb Oil (Piper Cubeba)

Cumin Oil (Cuminum Cyminum)

Curcuma Oil (Curcuma Longa)

Cypriol Oil (Cyperius Scariosus)

Cyperus Root Oil (Cyperus Rotundus)

Cypress Oil (Cypressus Sempervirens)

Davana Oil (Artemisia Pallens)

Dill Seed Oil (Anethum Graveolens)

Elemi Oil (Canarium Commune)

Estragon Oil (Artemisia Dracunculus)

Eucalyptus Citriodora Oil (Corymbia Citriodora)

Eucalyptus Globulus Oil (Eucalyptus Globulus)

Fennel Oil (Foeniculum Vulgare)

Galanga Oil (Alpinia Officinarum)

Galbanum Oil (Ferula Galbaniflua)

Geranium Oil (Pelargonium Graveolens)

Ginger Oil (Zingiber Officinale)

Grapefruit Oil (Citrus Paradisi)

Guaiacwood Oil (Bulnesia Sarmienti)

Gurjun Balsam Oil (Dipterocarpus Turbinatus)

Helichrysum Oil (Helichrysum Angustifolium)

Hyssop Oil (Hyssopus Officinalis)

Jasmin Absolute (Jasmin Grandiflorum)

Jatamansi Oil (Nardostachys Jatamansi)

Jonquil Absolute (Narcissus Joniquilla)

Juniper Berry Oil (Juniperus Communis)

Kaempferia Galanga Oil (Kaempferia Galanga)

Labdanum Resinoid (Cistus Ladaniferus)

Laurel Berry Oil (Laurus Nobilis)

Lavandin Super Oil (Lavandula Hybrida Var.)

Lavender Oil (Lavandula Angustufolia)

Lemongrass Oil (Cymbopogon Citratus)

Lemon Oil (Citrus Limomum)

Lemon Petitgrain Oil (Citrus Limomum)

Lime Oil (Citrus Aurantifolia)

Litsea Cubeba Oil (Litsea Cubeba)

Magnolia Absolute (Magnolia Grandiflora)

Mandarin Oil (Citrus Reticulata)

Mastic Resinoid (Pistacia Lentiscus)

Mate Absolute (Ilex Paraguayensis)

Melissa Oil (Melissa Officinalis)

Mentha Arvensis Oil (Mentha Arvensis)

Mentha Citrata Oil (Mentha Aquatica Var. Citrata)

Musk Deer Oil (Moschus Moschiferus)

Marigold Oil (Tagetes Glandulifera) Myrrh Resinoid (Commiphora Myrrha)

Myrtle Oil (Myrtus Communis)

Narcissus Absolute (Narcissus Poeticus)

Neroli Oil (Citrus Aurantium)

Orange Flower Water Absolute (Citrus Aurantium)

Nutmeg Oil (Myristica Fragrans Houtt.)

Oakmoss Absolute (Evernia Prunastri)

Olibanum Resinoid (Boswellia Carteri)

Opoponax Resinoid (Commiphora Erythrea)

Orange Flower Absolute (Citrus Aurantium)

Orange Oil (Citrus Sinensis)

Origanum Oil (Origanum Vulgare)

Orris Absolute (Iris Germanica)

Palmarosa Oil (Cymbopogon Martini)

Pandanus Oil (Pandanus Odoratissimus)

Parsley Seed Oil (Petroselinum Sativum)

Patchouli Oil (Pogostemon Cablin)

Pennyroyal Oil (Mentha Pulegium)

Peppermint Oil (Mentha Piperita)

Peru Balsam (Myroxylon Pereirae)

Petigrain Paraguay Oil (Citrus Aurantium, Subspecies Amara) Pimenta Berry Oil (Pimenta Officinalis) Pine Oil Sylvestre (Pinus Sylvestris) Rose Oil (Rosa Damascena) Rose Absolute (Rosa Damascena)

Rosemary Oil (Rosmarinus Officinalis) Sage Clary Oil (Salvia Sclarea) Sage Oil (Salvia Officinalis) Sandalwood East Indian Oil (Santalum Album) Schinus Molle Oil (Schinus Molle)

Spearmint Oil (Mentha Spicata) Star Anise Oil (Illicium Verum) Styrax Resinoid (Liquidambar Orientalis) Thyme Oil (Thymus Vulgaris) Tolu Balsam Oil (Myroxylon Toluiferum L. Oil)

Tonka Absolute (Dipteryx Odorata) Tuberose Absolute (Polyanthes Tuberosa) Valerian Oil (Valeriana Officinalis) Vanilla Absolute (Vanilla Planifolia) Verbena Oil (Lippia Citriodora)

Vetiver Oil (Vetiveria Zizanoides) Violet Leaf Absolute (Viola Odorata) Wintergreen Oil (Gaultheria Procumbens) Wormwood Oil (Artemisia Absinthium) Ylang-Ylang Oil (Cananga Odorata)

According to analysis of above essential oils, below volatile molecules selected for molecular docking.

The studied molecules can be seen at Table-1.

| Molecule | Cas No |
|---|---|
| 1-(1,4-dimethoxy-3-cylohexenyl)- | 43219-68-7 |
| ethanone | |
| 1,4-cineole | 470-67-7 |
| 1,8-cineole | 470-82-6 |
| 10-epi-gamma eudesmol | 15051-81-7 |
| 2-acetoxyfuranodiene | |
| 2-hydroxyfuranodiene | |
| 2-isopropyl phenol | 88-69-7 |
| 2-methyl hexanoic acid | 4536-23-6 |
| 2,3-dehydro-1,8-cineole | 92760-25-3 |
| 2-methoxyfuranadiene | 108376-98-3 |
| 2-methylbutyric acid | 116-53-0 |
| 3-propyl phenol | 621-27-2 |
| 3-phenyl propionic acid | 501-52-0 |
| 3-propylidene phthalide | 17369-59-4 |
| 8-hydroxyeremophilone | 5090-90-4 |
| acetyl isoeugenol | 93-29-8 |
| agarospirol | 1460-73-7 |
| alpha agarofuran | 5956-12-7. |
| alpha bergamotenol | 88034-74-6 |
| alpha bisabolene | 17627-44-0 |
| alpha bisabolol | 23089-26-1 |
| alpha bulnesene | 3691-11-0 |
| alpha cedrene | 469-61-4 |
| alpha copaene | 3856-25-5 |
| alpha cubebene | 17699-14-8 |
| alpha curcumene | 4176-17-4 |
| alpha cyperone | 473-08-5 |
| alpha farnesene | 502-61-4 |
| alpha fenchene | 471-84-1 |
| alpha guaiene | 3691-12-1. |
| alpha gurjunene | 489-40-7 |
| alpha patchoulene | 560-32-7 |
| alpha phellandrene | 99-83-2 |
| alpha pinene | 80-56-8 |
| alpha santalene | 512-61-8 |
| alpha santalol | 115-71-9 |
| alpha terpinene | 99-86-5 |
| alpha terpineol | 98-55-5 |
| alpha thujone | 546-80-5 |
| alpha vetivone | 15764-04-2 |
| alpha Zingeberene | 495-60-3 |
| Alpha-9-Aristolen1-ol | 34143-95-8 |
| alpha-ylangene | 14912-44-8 |
| Amyl salicylate | 2050-08-0 |
| anethole | 4180-23-8 |
| aromadendrene | 489-39-4 |
| benzoic acid | 65-85-0 |
| benzyl acetone | 2550-26-7 |
| beta agarofuran | 6040-08-0 |
| beta bisabolene | 495-61-4 |
| beta caryophyllene | 87-44-5 |
| beta cedrene | 548-28-1 |
| beta elemene | 515-13-9 |
| beta fenchene | 497-32-5 |
| beta gurjunene | 73464-47-8 |
| beta phellandrene | 555-10-2 |
| beta pinene | 18172-67-3 |
| beta santalol | 77-42-9 |
| beta selinene | 17066-67-0 |
| beta sesquiphellandrene | 20307-83-9 |
| beta thujone | 1125-12-8 |
| beta vetivone | 18444-79-6 |
| borneol | 507-70-0 |
| bornylene | 464-17-5 |
| butyric acid | 107-92-6 |
| camphene | 79-92-5 |
| camphor | 76-22-2 |
| capric acid | 334-48-5 |
| caproic acid | 142-62-1 |
| caprylic acid | 124-07-2 |
| carvacrole | 499-75-2 |
| carvacryl methyl ether | 6379-73-3 |
| caryophylenol | 4586-22-5 |
| cedrol | 77-53-2 |
| chavicol | 501-92-8 |
| cinnamic acid | 621-82-9 |
| cinnamyl benzoate | 5320-75-2 |
| cinnamyl cinnamate | 122-69-0 |
| cis-nuciferol | 78339-53-4 |
| cis-4-methoxy thujane | 1000371-47-5 |
| cis-lanceol | 10067-28-4 |
| cis-ocimene | 502-99-8 |
| citric acid | 77-92-9 |
| citronellol | 26489-01-0 |
| cuparene | 16982-00-6 |
| curzenone | 115526-32-4 |
| curzerene | 17910-09-7 |
| cyclohexyl carboxylic acid | 98-89-5 |
| cyperene | 2387-78-2 |
| cyperenol | 16981-80-9 |
| cyperotundone | 3466-15-7 |
| d-carvone | 2244-16-8 |
| delta cadinene | 483-76-1 |
| delta elemene | 20307-84-0 |
| delta selinene | 28624-23-9 |
| delta-3-carene | 13466-78-9 |
| dihydro karanone | 19624-46-5 |
| dimethyl anthranilate | 85-91-6 |
| elemol | 639-99-6 |
| epi-beta-santalol | 37172-32-0 |
| eremophilone | 562-23-2 |
| estragole | 140-67-0 |
| ethyl cinnamate | 103-36-6 |
| eugenol | 97-53-0 |
| eugenyl acetate | 93-28-7 |
| farnesol | 4602-84-0 |
| gamma gurjunene | 22567-17-5 |
| gamma himachellene | 53111-25-4 |
| gamma terpinene | 99-85-4 |
| gamma terpineol | 586-81-2 |
| geranic acid | 459-80-3 |
| geraniol | 106-24-1 |
| geranyl acetate | 105-87-3 |
| heptanoic acid | 111-14-8 |
| incensol | 22419-74-5 |
| indol | 120-72-9 |
| isopropyl trimethylcyclotetradecatrienol | 67901-02-2 |
| isoborneol | 464-45-9 |
| isobutyric acid | 79-31-2 |
| isomenthone | 491-07-6 |
| isopropyl cinnamate | 7780-06-5. |
| isovaleric acid | 503-74-2 |
| jatamansone | 1803-39-0 |
| jinkoheremol | 94201-17-9 |
| jinkohol | 66512-57-0 |
| karanone | |
| khusimone | 30557-76-7 |
| kusunol(valerianol) | 20489-45-6 |
| lactic acid | 50-21-5 |
| lauric acid | 143-07-7 |
| lavandulol | 58461-27-1 |
| l-dihydrofarnesal | 194934-66-2 |
| l-dihydrofarnesol | 51411-24-6 |
| limonene | 5889-27-5 |
| linalol | 78-70-6 |
| linalyl acetate | 115-95-7 |
| linalyl cinnamate | 78-37-5 |
| lindestrene | 2221-88-7 |
| longifolene | 475-20-7 |
| menthol | 2216-51-5 |
| menthone | 14073-97-3 |
| methl cinnamate | 103-26-4 |
| methyl anthranilate | 134-20-3 |
| methyl eugenol | 93-15-2 |
| methyl isoeugenol | 93-16-3 |
| methyl salicylate | 119-36-8 |
| muskaton | 1209-91-2 |
| myrcene | 123-35-3 |
| myristic acid | 544-63-8 |
| nerol | 106-25-2 |
| neroloxide | 1786-08-9 |
| nootkatone | 4674-50-4 |
| oxo-agarospirol | 23811-08-7 |
| oxyphenylone | 5471-51-2 |
| p-cymene | 99-87-6 |
| patchulol | 5986-55-0 |
| p-cresol | 106-44-5 |
| pelargonic acid | 112-05-0 |
| p-ethyl phenol | 123-07-9 |
| phenylacetic acid | 103-82-2 |
| phenylethyl alcohol | 60-12-8 |
| phenylpropy cinnamate | 122-68-9 |
| p-mentha-3,8-diene | 586-67-4 |
| p-menthadien-7-al | 1197-15-5 |
| pulegone | 89-82-7 |
| roduntene | 65128-08-7 |
| roduntone | 18374-76-0 |
| rose oxide | 16409-43-1 |
| sabinene | 3387-41-5 |
| sabinyl acetate | 3536-54-7 |
| salicylaldehyde | 90-02-8 |
| santalol | 11031-45-1 |
| santene | 529-16-8 |
| scatole | 83-34-1 |
| sclareol | 515-03-7 |
| seychellene | 20085-93-2 |
| spirojatamol | 128487-46-7 |
| t-2-hexenoic acid | 13419-69-7 |
| t-3-hexenoic acid | 1577-18-0 |
| terpinen-4-ol | 562-74-3 |
| terpinolene | 586-62-9 |
| thujadiene | 36262-09-6 |
| thujopsene | 470-40-6 |
| thymol | 89-83-8 |
| thymyl methyl ether | 1076-56-8 |
| trans ocimene | 6874-44-8 |
| trans-alpha bergamotene | 13474-59-4 |
| tricyclene | 508-32-7 |
| undecanoic acid | 112-37-8 |
| undeylenic acid | 112-38-9 |
| valerenal | 4176-16-3 |
| valeric acid | 109-52-4 |
| vanillin | 121-33-5 |
| verbenene | 4080-46-0 |
| vetiveryl acetate | 117-98-6 |
| vetyvalone | |
| zingerone | 122-48-5 |
| ziza-6(13)-en-3-alpha-ol | |
| ziza-6(13)-en-3-one | |

Analysis of docking studies, carried by two different software and total of three different methods showed that several of the tested molecules showed low binding affinities, as good as or better than the drug candidate molecules against SARS-CoV-2, presented in the current literature. This finding suggest that the molecules found in this study are good candidates to bind the selected target molecules MPro and BRD2. Following the virtual screening and lead identification studies presented in this study, lead optimization and clinical studies could initiate the discovery of a new drug(s) that can potentially prevent and/or cure SARS-CoV-2 infection. According to our in-silico study, the following essential oil molecules (Table-2) shows best binding affinities towards SARS-CoV-2 virus.

TABLE 2

| Molecule | Cas No | BRD2 | MPro |
|---|---|---|---|
| 10-epi-gamma eudesmol | 15051-81-7 | −7.2 | −7.0 |
| 2_acetoxyfuranodiene | | −8.5 | −6.7 |
| 2_hydroxyfuranodiene | | −7.9 | −7.3 |
| 2-methoxyfuranodiene | 108376-98-3 | −9.8 | −7.2 |
| agarospirol | 1460-73-7 | −7.0 | −6.4 |
| alpha bisabolene | 17627-44-0 | −7.8 | −6.4 |
| alpha bulnesene | 3691-11-0 | −8.4 | −5.5 |
| alpha copaene | 3856-25-5 | −6.7 | −6.0 |
| alpha cubebene | 17699-14-8 | −7.4 | −6.2 |
| alpha curcumene | 4176-17-4 | −6.8 | −5.5 |
| alpha cyperone | 473-08-5 | −9.3 | −5.7 |
| alpha guaiene | 3691-12-1. | −8.4 | −6.2 |
| alpha gurjunene | 489-40-7 | −7.3 | −5.7 |
| alpha patchoulene | 560-32-7 | −6.4 | −6.0 |
| alpha Zingeberene | 495-60-3 | −7.2 | −5.6 |
| alpha-9-Aristolen1-ol | 34143-95-8 | −7.6 | −6.2 |
| aromadendrene | 489-39-4 | −7.9 | −5.9 |
| benzyl benzoate | 120-51-4 | −8.1 | −6.3 |
| benzyl salicylate | 118-58-1 | −8.1 | −6.9 |
| beta santalol | 77-42-9 | −7.1 | −5.5 |
| beta selinene | 17066-67-0 | −8.5 | −6.4 |
| beta sesquiphellandrene | 20307-83-9 | −6.9 | −6.2 |
| beta vetivone | 18444-79-6 | −8.4 | −6.1 |
| caryophylenol | 4586-22-5 | −8.5 | −5.9 |
| cinnamyl benzoate | 5320-75-2 | −8.4 | −6.0 |
| cinnamyl cinnamate | 122-69-0 | −9.2 | −6.8 |
| curzenone | 115526-32-4 | −7.6 | −6.3 |
| curzerene | 17910-09-7 | −7.3 | −5.9 |
| cyperene | 2387-78-2 | −6.8 | −5.3 |
| cyperotundone | 3466-15-7 | −7.0 | −5.9 |
| delta cadinene | 483-76-1 | −7.4 | −5.5 |
| dihydroambrettolide | 109-29-5 | −8.0 | −7.1 |
| dihydro karanone | 19624-46-5 | −8.5 | −6.6 |
| elemol | 639-99-6 | −6.6 | −5.1 |
| epi-beta-santalol | 37172-32-0 | −7.4 | −6.1 |
| eremophilone | 562-23-2 | −7.6 | −6.0 |
| galaxolide | 1222-05-5 | −8.6 | −7.4 |
| gamma gurjunene | 22567-17-5 | −7.0 | −5.3 |
| incensol | 22419-74-5 | −8.2 | −6.8 |
| isopropyl | 67921-02-2 | −8.2 | −6.6 |
| trimethylcyclotetradecatrienol | | | |
| jinkoheremol | 94201-17-9 | −7.4 | −6.3 |
| karanone | | −7.9 | −5.9 |
| kusunol(valerianol) | 20489-45-6 | −7.7 | −6.5 |
| Lindestrene | 2221-88-7 | −7.7 | −6.3 |
| l-muscone | 10403-00-6 | −6.7 | −6.4 |
| muskaton | 1209-91-2 | −7.5 | −5.7 |
| nootkatone | 4674-50-4 | −8.7 | −6.6 |
| patchulol | 5986-55-0 | −7.2 | −6.7 |
| phenylpropy cinnamate | 122-68-9 | −8.7 | −5.5 |
| roduntone | 18374-76-0 | −8.0 | −6.4 |
| sclareol | 515-03-7 | −7.5 | −8.1 |
| seychellene | 20085-93-2 | −6.5 | −5.1 |
| valerenal | 4176-16-3 | −7.6 | −6.1 |
| vertofix | 32388-55-9 | −6.9 | −7.2 |
| vetiveryl acetate | 117-98-6 | −8.1 | −6.6 |

TABLE 3
Molecular Structures of Table-2 Molecules

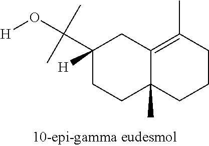 [SMILES: CC1=C2C[C@@H](C(C)(C)O)CC[C@@]2(C)CCC1]

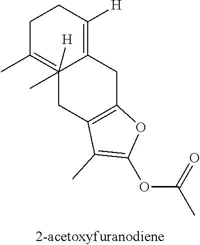

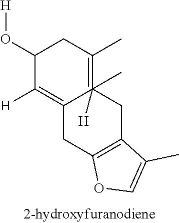

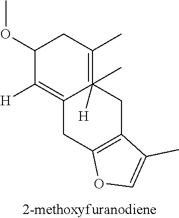

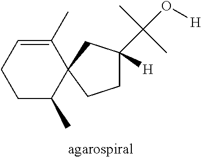 [SMILES: CC1=CCC[C@H](C)[C@@]12CC[C@@H](C(C)(C)O)C2]

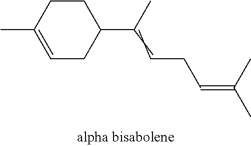

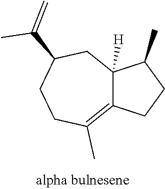

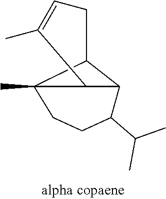

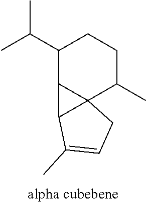

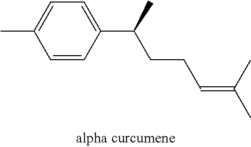

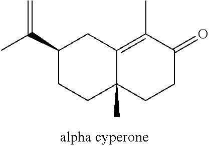 [SMILES: C=C(C)[C@@H]1CC[C@@]2(C)CCC(=O)C(C)=C2C1]

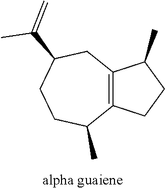

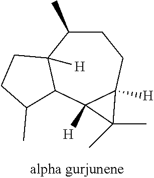

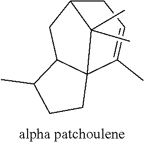

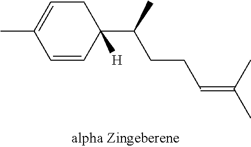

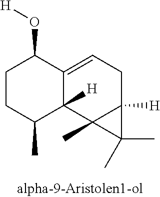

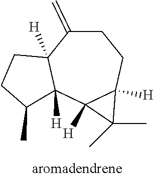

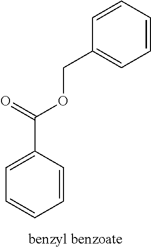 [SMILES: O=C(OCc1ccccc1)c1ccccc1]

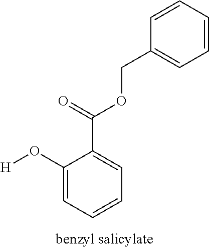 [SMILES: O=C(OCc1ccccc1)c1ccccc1O]

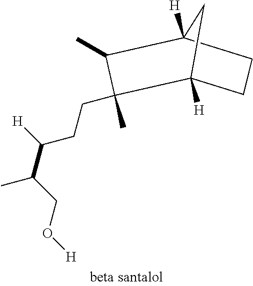

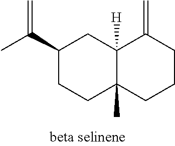

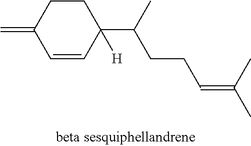

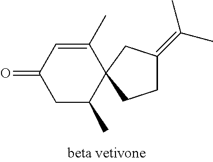 [SMILES: CC1=CC(=O)C[C@H](C)[C@]12CCC(=C(C)C)C2]

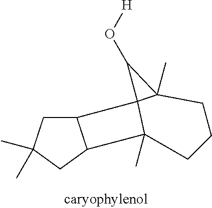 [SMILES: CC1(C)CC2C(C1)C13C(O)C1(C)CCCC23C]

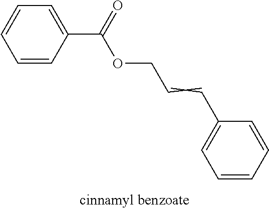 [SMILES: O=C(OCC=Cc1ccccc1)c1ccccc1]

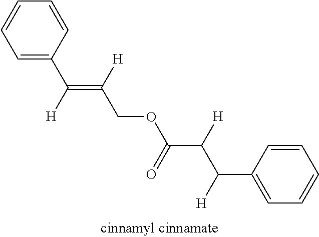 [SMILES: O=C(CCc1ccccc1)OC/C=C/c1ccccc1]

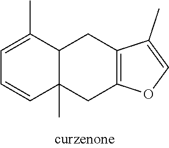 [SMILES: CC1=CC=CC2(C)Cc3occ(C)c3CC12]

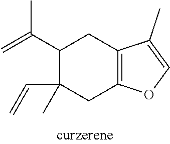 [SMILES: C=CC1(C)Cc2occ(C)c2CC1C(=C)C]

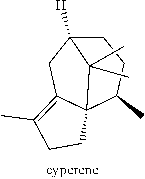

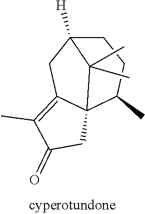

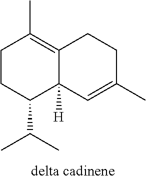

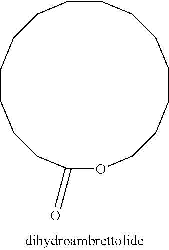 [SMILES: O=C1CCCCCCCCCCCCCCO1]

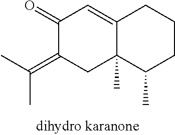 [SMILES: CC(C)=C1C[C@@]2(C)C(=CC1=O)CCC[C@@H]2C]

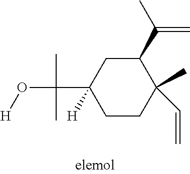 [SMILES: C=C[C@]1(C)CC[C@@H](C(C)(C)O)C[C@H]1C(=C)C]

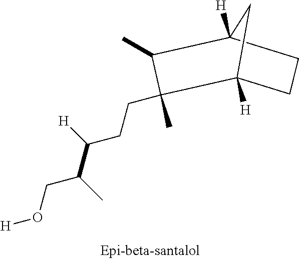

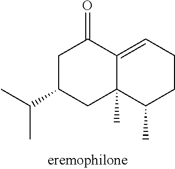 [SMILES: CC(C)[C@@H]1CC(=O)C2=CCC[C@H](C)[C@@]2(C)C1]

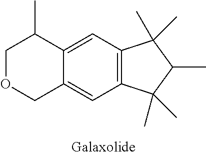 [SMILES: CC1COCc2cc3c(cc21)C(C)(C)C(C)C3(C)C]

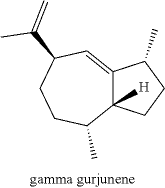

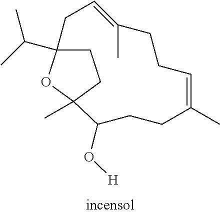 [SMILES: C/C1=C/CC/C(C)=C/CC2(C(C)C)CCC(C)(O2)C(O)CC1]

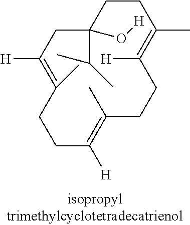 [SMILES: C/C1=C\CC/C(C)=C/CC(O)(C(C)C)CC/C(C)=C/CC1]

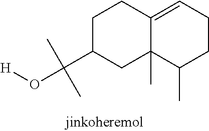 [SMILES: CC1CCC=C2CCC(C(C)(C)O)CC21C]

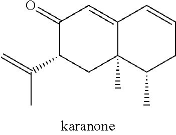 [SMILES: C=C(C)[C@H]1C[C@@]2(C)C(=CC1=O)C=CC[C@@H]2C]

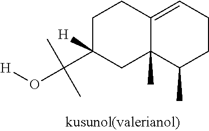 [SMILES: C[C@@H]1CCC=C2CC[C@@H](C(C)(C)O)C[C@]21C]

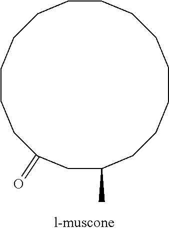 [SMILES: C[C@@H]1CCCCCCCCCCCCCC(=O)C1]

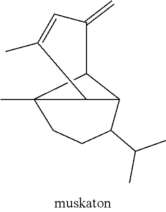 [SMILES: C=C1C=C(C)C2C3C(C(C)C)CCC2(C)C13]

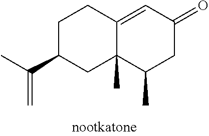 [SMILES: C=C(C)[C@H]1CCC2=CC(=O)C[C@@H](C)[C@]2(C)C1]

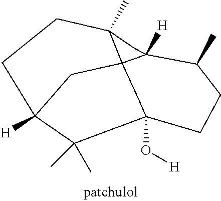

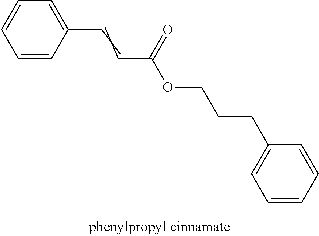 [SMILES: O=C(C=Cc1ccccc1)OCCCc1ccccc1]

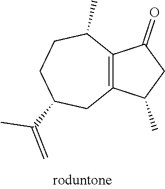 [SMILES: C=C(C)[C@@H]1CC[C@H](C)C2=C(C1)[C@@H](C)CC2=O]

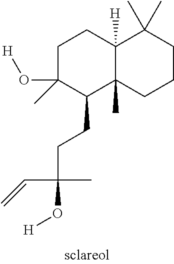 [SMILES: C=C[C@](C)(O)CC[C@H]1C(C)(O)CC[C@H]2C(C)(C)CCC[C@@]21C]

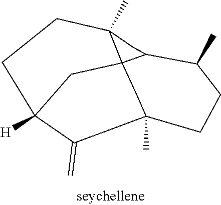

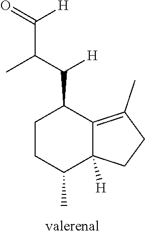 [SMILES: CC1=C2[C@H](CC(C)C=O)CC[C@@H](C)[C@@H]2CC1]

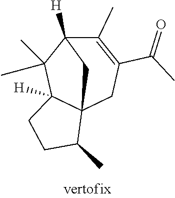 [SMILES: CC(=O)C1=C(C)[C@@H]2C[C@@]3(C1)[C@@H](C)CC[C@H]3C2(C)C]

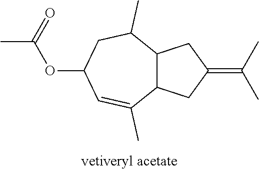 [SMILES: CC(=O)OC1C=C(C)C2CC(=C(C)C)CC2C(C)C1]

Whereas 2-methoxyfuranodiene, 2-acetoxyfuranodiene, 2-hydroxyfuranodiene, lindestrene, curzerene are parts of Myrrhe oil (Commiphora Myrrha), nootkatone is a part of Grapefruit oil (Citrus Paradisi), sclareol is a part of Sage Clary Absolute (Salvia Sclare), beta caryophyllene, rotundone, alpha bulnesene, alpha guaiene are parts of Patchouly Oil (Pogostemon Cablin), agorospirol, alpha agarofuran, dihydro karanone, karanone, jinkoheremol are the parts of Agarwood Oil (Aquilaria malacsensis, Aquilaria Crassna, Aquilaria sinensis), gamma gurjunene, beta gurjunene, alpha gurjunene, aromadendrene, delta cadinene, delta elemene, beta elemene, alpha cubebene, alpha ylangene are the parts of Gurjun Balsam Oil (Dipterocarpus Turbinatus), incensol and isopropyl trimethylcyclotetradecatrienol are the parts of Olibanum Resinoid (Boswellia Carteri), alpha cyperone, cyperene, cyprotundone, muskaton are the part of Cypriol Oil (Cyperius Scariosus), phenylpropyl cinnamate, cinnamyl cinnamate, benzyl cinnamate, cinnamyl benzoate, benzyl benzoate, benzyl salicylate are the parts of Styrax Oil or Styrax Resinoid (Liquidambar Orientelis), alpha copaene and alpha-cubebene are the parts of Copaiba Balsam Oil (Copaifera Reticulata), khusimon, beta vetivone, alpha zingiberene, beta selinene, beta sesquiphellendrene, beta bisabolene, alpha bisabolene, alpha curcumene, alpha farnesene are parts of Ginger Oil (Zingiber Officinale), vetiveryl acetate is derivative of acetylated vetiver oil,also khusimon and beta-vetivone are the parts of Vetiver Oil (Vetiveria Zizanoides), L-Muscone is a component of Musc Deer Oil (purely synthesized is available), alpha bergamotenol, cis-nuciferol, cis-lanceol, alpha bisabolol are the parts of Sandalwood Oil (Santalum Album), Jatamansone, valerianol, valerenal are the parts of Jatamansi Oil, alpha curcumene is a part of Curcuma Oil (Curcuma Longa), Galaxolide is a synthetic fragrance molecule, dihydroambrettolide is a synthetic fragrance molecule, vertofix (acetylated alpha cedrene and thujopsene) is a Cedarwood Oil (Juniperus Virginiana) derivative.

Although, the molecules in Table-2 could be extracted from corresponding essential oils or could be synthesized. It is much easier, cheaper and practical to use the essential oil whereas the molecule has been found. Mixing these essential oils create a synergetic effect in combating virus and could be much more effective. Once viral effective essential oils are combined with anti-bacterial essential oils which are used as traditionally phenomena treatment would be a complete potential preventing agent.

Using essential oil vapors as inhaled antiseptics would be effective against combinations of viruses, bacteria and perhaps fungi that could form associated, or symbiotic relationships, resulting in complex infections that might be responsible for SARS-CoV-2 (Covid-19). An inhaled antiseptic approach is ideal when the nature of the pathogen is unknown and/or the pathogen mutates rapidly. The essential oil vapors can be provided immediately to humans in the form of simple hand-held inhaler devices.

Most essential oils are fat soluble. Therefore, these oils permeate the lung tissue. After inhalation, some of the vapors enter the blood stream. These oils are thought to cross the blood-brain boundary and may possibly eradicate pathogens elsewhere in the body. According to the literature, there is little chance for humans overdosing on inhaling essential oil vapors. In about two hours after inhalation, the vapors have normally been exhaled back out of the body--primarily from the lungs.

Selected essential oils, smell very good to humans, and the patients are therefore likely to repetitively inhale the vapors to suitably prevent, treat, or cure infections from pathogens causing SARS-CoV-2. It is likely necessary to repetitively inhale the essential oil vapors every two hours or so to prevent infections caused by a nearby infected individual who may be coughing, sneezing, etc. Infections of pathogens causing SARS-CoV-2 may be made more severe in the presence of other infections such as bronchitis, TB, or some other form of pneumonia in the patient. Therefore, a broad antiseptic approach is desirable. If such an inhaled antiseptic approach appeared to “help”, it could be implemented immediately giving more time to come up with other therapies, perhaps including immunization therapies. Inhaled antiseptic essential oil vapors have an immediate impact in the lungs, whereas digested pills take relatively longer, and immunization can still take longer periods of time.

Definitions

As used herein, the recited terms have the following meanings. All other terms and phrases used in this specification have their ordinary meanings as one of skill in the art would understand after review of this disclosure.

As used herein, the terms “EOs” or “essential oils” refer to aromatic, volatile liquids extracted from organic material, such as plants. Fragrance substances are mixtures of natural essential oils and synthetic organic odorous compounds with characteristic, usually pleasant, odors. They are used in perfumes and perfumed cosmetic products, in detergents, soaps, fabric softeners, air care, incense and other household products where fragrance may be used to mask unpleasant odors from raw materials and give a pleasant smell [12-16]. These substances are also used in aromatherapy and other products as topical medicaments for their antiseptic, antibacterial, antifungal, and antiviral properties.

Essential oils can be derived from the flowers, fruits, seeds, leaves, stalks, barks, roots, and rhizomes of sources. As used herein, “plants” and “plant derivatives” can refer to any portion of a growing plant, including the roots, stems, stalks, leaves, branches, berries, seeds, flowers, fruits, bark, wood, rhizomes, resins, and the like.

As used herein, the term “agitate” refers to exerting an outside force on a material, such as stirring, shaking, or vibrating. A vessel can be agitated by turning, tipping, shaking, etc. A paddle or stirrer can be utilized within a vessel to agitate.

As used herein, the term “emulsion” refers to a system containing two or more liquids, in which at least one liquid is not substantially soluble or miscible in at least one other liquid. In an emulsion, one liquid, the “dispersed phase”, is dispersed throughout a second liquid, the “continuous phase”, and is often present as a fine dispersion of droplets. An EO may be emulsified or substantially emulsified within a carrier medium, such as water. In this example, the water is the continuous phase, and the EO is the dispersed phase present as a dispersion of droplets. An emulsion can optionally include an emulsifier and/or stabilizer, which can encourage the formation of the droplets by the dispersed phase, maintain the size or shape of the dispersed phase droplets, assist in reducing or reduce the size of the dispersed phase droplets, or combinations thereof. Emulsions can significantly increase the surface area of a dispersed phase. Some emulsions can further comprise dispersed insoluble particles such as solid carriers, mineral chelates, mineral salts, or the like. A low droplet size of a dispersed phase can advantageously aid in the dispersion of insoluble particles throughout the continuous phase.

As used herein, the term “emulsifier” refers to a substance that stabilizes an emulsion. The emulsifier can utilize physical properties, chemical properties, or utilize both physical and chemical properties to interact with one or more substances of an emulsion.

As used herein, “carrier” refers to a substance that physically or chemically binds or combines with a target or active substance to facilitate the use, storage, or application of the target or active substance. Carriers are often inert materials, but can also include non-inert materials when compatible with the target or active substances. Examples of carriers include, but are not limited to, water for compositions that benefit from a liquid carrier, or diatomaceous earth or limestone for compositions that benefit from a solid carrier.

The disclosure herein indicates the efficacy of compositions comprising a plurality of EOs which provide a synergistic effect beyond EOs utilized in isolation. Further, EO compositions provided herein do not exhibit antagonistic effect between EO moieties within a composition. An EO composition generally includes an EO fraction and optionally one or more additional components. The ratio of the EO fraction to the one or more additional components in a composition can depend on several factors such as administration method, and the nutritional/health needs and/or palate of a consuming subject, among others. In many embodiments, a consuming subject comprises an animal or a human. Compositions can comprise additional components including carriers, emulsifiers, and stabilizers, among others. Compositions can be in the form of an emulsion.

Embodiments of EO compositions provided herein are effective against enveloped viruses, especially SARS-CoV-2. EO compositions can impair, disable, or destroy viral envelopes, thereby preventing the virus from identifying host cells, fusing to host cells, or protecting the viral body against a host immune system. As a result of these non-limiting proposed antiviral mechanisms, the compositions provided herein are generally effective against most or all strains of SARS-CoV-2. EO compositions provided herein can be administered in combination with vaccines and pharmaceuticals. The EO compositions described herein further provide indirect antiviral benefits. For example, EOs are known to be antibacterial and antifungal and have low cytotoxicity, and accordingly maintain or increase immune system strength.

The EO fractions of embodiments disclosed herein are configured to provide an antiviral effect with low toxicity to the host subject or system. Further, such low toxicity is paired with an effect which generally enhances the health and immune system of a host subject, thereby providing a second antiviral effect stemming from the immune system of the host in addition to the antiviral effect of the EO composition.

EO compositions as provided herein contain EOs derived from plants (i.e., “natural” EOs) and additionally or alternatively their synthetic analogues. Many embodiments comprise a combination of EOs. Some embodiments comprise a combination of natural and synthetic materials. [17]

Throughout the evolutionary history of life on Earth, essential oils in plants and trees have been theorized to evolve with viruses, bacteria and fungi to protect plants and trees from viral, bacterial and fungal infections.

### EXAMPLES

Preparation of an anti-viral/anti-bacterial compositions:

The anti-viral/anti-bacterial composition was prepared by admixing the following ingredients:

### Formula A (Antibacterial/Antiviral Composition)

| | PERCENTAGE |
|---|---|
| INGREDIENTS | (%) |
| BENZYL SALICYLATE | 5.00 |
| CINNAMON BARK OIL | 0.25 |
| CURCUMA OIL | 1.00 |
| EUCALYPTUS GLOBULUS OIL | 60.00 |
| TEA TREE OIL | 25.00 |
| GINGER OIL | 2.00 |
| ROSEMARY OIL | 5.00 |
| BENZYL BENZOATE | 1.75 |
| TOTAL | 100.00 |

The anti-viral composition was prepared by admixing the following ingredients:

### Formula B (Antiviral Composition)

| | PERCENTAGE |
|---|---|
| INGREDIENTS | (%) |
| JATAMANSI OIL | 1.00 |
| GURJUN BALSAM OIL | 5.00 |
| STYRAX RESINOID | 1.00 |
| STYRAX OIL | 1.50 |
| MYRRHE RESINOID | 20.00 |
| MYRRHE OIL | 20.00 |
| AGARWOOD OIL | 1.00 |
| COPAIBA BALSAM OIL | 1.00 |
| GINGER OIL | 0.50 |
| CYPRIOL OIL | 2.50 |
| SANDALWOOD OIL EAST INDIAN | 1.00 |
| GRAPEFRUIT OIL | 2.50 |
| SALVIA SCLAREE ABSOLUTE | 20.00 |
| VETIVERYL ACETATE | 5.00 |
| L-MUSCONE | 0.20 |
| BENZYL CINNAMATE | 2.50 |
| OLIBANUM RESINOID | 10.00 |
| PATCHOULY OIL | 2.50 |
| NOOTKATONE | 2.50 |
| BENZYL BENZOATE | 0.30 |
| TOTAL | 100.00 |

The anti-viral composition was prepared by admixing the following ingredients:

### Formula C (Antiviral Composition)

| | PERCENTAGE |
|---|---|
| INGREDIENTS | (%) |
| GURJUN BALSAM OIL | 10.00 |
| MYRRHE RESINOID | 25.00 |
| AGARWOOD OIL | 5.00 |
| CYPRIOL OIL | 2.50 |
| SALVIA SCLAREE ABSOLUTE | 2.00 |
| OLIBANUM RESINOID | 10.00 |
| BENZYL BENZOATE | 45.50 |
| TOTAL | 100.00 |

The anti-viral/anti-bacterial compositions were prepared by admixing the following ingredients:

### Formula D (Antibacterial/Antiviral Composition)

| | PERCENTAGE |
|---|---|
| INGREDIENTS | (%) |
| FORMULA A | 50.00 |
| FORMULA C | 50.00 |
| TOTAL | 100.00 |

### Formula E (Antibacterial/Antiviral Composition)

| | PERCENTAGE |
|---|---|
| INGREDIENTS | (%) |
| FORMULA A | 50.00 |
| FORMULA B | 50.00 |
| TOTAL | 100.00 |

### Formula F (Antibacterial/Antiviral Composition)

| | PERCENTAGE |
|---|---|
| INGREDIENTS | (%) |
| FORMULA A | 40.00 |
| FORMULA B | 40.00 |
| BENZYL BENZOATE | 20.00 |
| TOTAL | 100.00 |

### Formula G (Antibacterial/Antiviral Composition)

| | PERCENTAGE |
|---|---|
| INGREDIENTS | (%) |
| FORMULA A | 25.00 |
| FORMULA B | 25.00 |
| BENZYL BENZOATE | 50.00 |
| TOTAL | 100.00 |

Besides of above compositions Myrrhe essential oil and resinoid, Salvia Sclaree Absolute, Olibanum Resinoid could be used alone or in combination with dilution in benzyl benzoate at consumer products.

Formulation Examples

### Example 1—Hand Sanitizer Formulation

| | % BY |
|---|---|
| INGREDIENTS | WEIGHT |
| DEIONIZED WATER | 29.1 |
| CARBOPOL ULTREZ 20¹⁾ | 0.2 |
| GLYCERIN²⁾ | 1 |
| DENATURATED ALCOHOL | 62.7 |
| TRIETHANOLAMINE % 10 ³⁾ | 2 |
| FORMULA E (ANTIBACTERIAL/ | 5 |
| ANTIVIRAL COMPOSITION) | |
| TOTAL | 100 |

¹⁾Carbopol Ultrez 20, Origin: Lubrizol
²⁾Glycerin, Origin: Evyap A. [symbol] .
³⁾ Triethanolamine, Origin: Sasol Performance Chemicals

Hand sanitizer was prepared by dispersed in water and Carbopol Ultrez 20 without agitation. Agitation is started once polymer was wetted. Then, glycerin was added. Alcohol is dispersed slowly into the lotion. 10% Triethanolamine solution is added to solution and finally anti-bacterial/anti-viral composition Formula E is added.

To provide the benefits of the present invention in a hand sanitizer form, the liquid composition produced in accordance with Example-1, may be packaged in a typical PET bottle with a flip-top cap. Liquid is dispensed to the hands in an amount to ensure complete wetting. Employing this method affords immediate anti-viral action and provides long-lasting activity after drying.

### Example 2— Pocket Size Self Atomizer Anti-bacterial/Anti-Viral Cologne Formulation

| | % BY |
|---|---|
| INGREDIENTS | WEIGHT |
| DEIONIZED WATER | 16.70 |
| DENATURATED ALCOHOL | 78.30 |
| FORMULA D (ANTIBACTERIAL/ | 5 |
| ANTIVIRAL COMPOSITION) | |
| TOTAL | 100 |

Cologne was prepared by mixing deionized water and denaturated alcohol to obtain 80° Alcohol. The anti-bacterial/anti-viral cologne was prepared by adding 5% Formula D by weight under gentle shaking.

To provide the benefits of the present invention in a cologne form, the emulsion produced in accordance with example-2, may be packaged in a typical PET bottle with a trigger at top. Liquid is sprayed to the hands in an amount to ensure complete wetting. Employing this method affords immediate anti-viral action and provides long-lasting activity after drying.

### Example 3—Anti-bacterial/Anti-Viral Shampoo Formulation

| | % BY |
|---|---|
| INGREDIENTS | WEIGHT |
| A. | |
| DEIONIZED WATER | 77.2 |
| TEXAPON N 70¹⁾ | 14 |
| KOMPERLAN KD¹⁾ | 2 |
| KATHON CG²⁾ | 0.05 |
| B. | |
| DEHYTON PK45¹⁾ | 3 |
| GLYCERIN³⁾ | 1 |
| D-PANTHENOL 75 W¹⁾ | 0.2 |
| C. | |
| CITRIC ACID⁴⁾ | 0.05 |
| D. | |
| SODIUM CHLORIDE⁵⁾ | 2 |
| E. | |
| FORMULA G (ANTIBACTERIAL/ | 0.5 |
| ANTIVIRAL COMPOSITION) | |
| TOTAL | 100 |

¹⁾TEXAPON N 70, KOMPERLAN KD. DEHYTON PK 45, D-PANTHENOL 75 W, Origin: BASF
²⁾Kathon CG, Origin: Dow Chemicals
³⁾Glycerin, Origin: Evyap A. [symbol] .
⁴⁾Citric Acid, Origin: Merck
⁵⁾Sodium Chloride, Table salt

The shampoo was prepared by dispersed in water and Texapon N 70, Komperlan KD and Kathon CG. Mixture was heated to 50-55° C. while stirring. Phase B (Dehyton PK 45, Glycerin and D-Panthenol 75 W) was prepared and added once Phase A was homogeneous. Then, citric acid is added into the solution. Final viscosity was adjusted with NaCl. The anti-bacterial/anti-viral shampoo was prepared by adding Formula G, 0.5% by weight, under gentle shaking.

To provide the benefits of the present invention in a shampoo form, the liquid composition produced in accordance with example-3, may be packaged in a typical PET bottle with a flip-top cap. Shampoo is poured to the hands in an amount to ensure complete cleaning of hair. Rinse the hair and repeat same steps if necessary. Employing this method affords immediate anti-viral action and provides long-lasting activity after washing and drying hair.

### Example 4—Anti-bacterial/Anti-Viral Electrical Diffuser Formulation

To provide the benefits of the present invention in an electrical diffuser form, anti-bacterial/anti-viral Formula G, 50% by weight with suitable solvents or carrier oils, added in electrical diffuser machine. The machine is adjusted according to the requirement. Employing this method affords immediate anti-viral action and provides long-lasting activity in regular application.

Example 5—Anti-bacterial/Anti-viral Reed Diffuser Formulation

Reed diffusers deliver anti-bacterial/anti-viral composition to the atmosphere through the processes of adsorption and then diffusion. To provide the benefits of the present invention in a reed diffuser form, 20% of Formula G is added to 80° Alcohol (produced in accordance with example-2). Such present formulation was blended together to yield 50 grams of reed diffuser oil, which was then placed in a 75 ml glass bottle. Then, 20cm, 4 reeds were placed in such bottle. Wood, plant-based, or cellulose fiber materials are used as reed due to their oil absorbing characteristic. Based on diffusion principles, the oil is conducted, via a capillary action, to surfaces where the oil can evaporate. The evaporation rate was then monitored by weight loss over the time. Employing this method affords anti-viral action by time as anti-bacterial/anti-viral composition diffuses to the air and provides long-lasting activity in regular application.

### Example 6—Anti-bacterial/Anti-Viral Air Freshner & Sanitizer Formulation

To provide the benefits of the present invention in an air freshener form, 5% of the anti-bacterial/anti-viral Formula G is added to 80° Alcohol (produced in accordance with example-2). The mixture is filled in an appropriate PET bottle with a trigger at top. Employing this method affords immediate anti-viral action and provides long-lasting activity in regular application.

### Example 7—Anti-bacterial/Anti-Viral Air Nebulizer Formulation

To provide the benefits of the present invention in an air nebulizer form, 10-15 drops of anti-bacterial/anti-viral Formula F is added to water in air nebulizer machine. The machine is adjusted according to the requirement. Employing this method affords immediate anti-viral action and provides long-lasting activity in regular application.

### Example 8 Anti-bacterial/Anti-Viral Hand Cream Formulation

| | % BY |
|---|---|
| INGREDIENTS | WEIGHT |
| A. | |
| DEIONIZED WATER | 77.14 |
| CARBOPOL ULTREZ 21¹⁾ | 0.30 |
| GLYCERIN²⁾ | 4.00 |
| KATHON CG³⁾ | 0.10 |
| B. | |
| EMULDAC AS-25⁴⁾ | 1.55 |
| CERASYNT SD⁵⁾ | 1.41 |
| CRODACOL C90⁶⁾ | 3.00 |
| WHITE MINERAL OIL LX-15⁷⁾ | 2.00 |
| MYRITOL 318⁸⁾ | 3.00 |
| ISOPROPYL MYRISTATE⁹⁾ | 3.00 |
| XIAMETER ™ PMX-200 SILICONE FLUID 350 CS³⁾ | 0.50 |
| C. | |
| TRIETHANOLAMINE SOLUTION (% 10) ⁴⁾ | 3.00 |
| D. | |
| FORMULA D (ANTIBACTERIAL/ANTIVIRAL | 0.5 |
| COMPOSITION) | |
| TOTAL | 100 |

¹⁾Carbopol Ultrez 21, Origin: Lubrizol
²⁾Glycerin, Origin: Evyap A. [symbol] .
³⁾Kathon CG, Origin: Dow Chemicals
⁴⁾Emuldac AS-25, Origin: Sasol
⁵⁾Cerasynt SD, Origin: Ashland
⁶⁾Crodacol C90, Origin: Croda
⁷⁾White Mineral Oil LX-15, Origin: Eastern Patroleum
⁸⁾Myritol 318, Origin: BASF
⁹⁾Isopropyl Myristate, Origin: Ventos

The hand cream was prepared by dispersed in water and Carbopol Ultrez 21 without agitation. As the polymer is wetted, agitation is started and mixture was heated to 50-55°. As the mixture is homogeneous Carbapol is added first, after that Glycerin and Cathon CG are added. Phase B was prepared and heated till the solution is melted. Phase B is added with agitation, once Phase A was homogeneous. Then, triethanolamine is added into the emulsion to neutralize and mixed. The anti-bacterial/anti-viral hand cream was prepared by adding Formula D, 0.5% by weight, under gentle agitation.

Cream is applied to hands as needed. Employing this method affords immediate anti-viral action and provides long-lasting activity after drying.

### Example 9 Anti-bacterial/Anti-Viral Wet Wipe Formulation

| | % BY |
|---|---|
| INGREDIENTS | WEIGHT |
| A. | |
| DEIONIZED WATER | 94.77 |
| PROPYLENE GLYCOL¹⁾ | 1.00 |
| PLANTACARE 818 UP¹⁾ | 0.50 |
| EUXYL PE 9010²⁾ | 1.00 |
| EMULGIN CO 40¹⁾ | 0.50 |
| B. | |
| EDETA BX POWDER¹⁾ | 0.20 |
| C. | |
| CITRIC ACID | 0.03 |
| D. | |
| FORMULA E (ANTIBACTERIAL/ | 2 |
| ANTIVIRAL COMPOSITION) | |
| TOTAL | 100 |

¹⁾Propylene Glycol, Plantacare 818 UP, Emulgine CO 40, Edata BX Powder, Origin: BASF
²⁾Euxyl PE 9010, Origin:Schulke INC.

The lotion is produced by mixing all ingredients in Phase A until the solution is clear and adding Edeta Bx Powder and Citric acid on the clear solution. The anti-bacterial/anti-viral wet wipe was prepared by adding Formula E, 2% by weight, under gentle shaking.

This solution may be used to produce a wet wipe product for topical cleaning and/or sanitizing of skin and/or hard surfaces. Such a product is made by saturating a paper or cloth substrate with the liquid composition prepared in accordance with the previous section. The level of saturation depends upon the substrate in which incorporation of the antiviral composition is desired. A 5″×8″ hand wipe paper made from 40-60 grams per square meter spun-lace non-woven material may be saturated with about 1-3 grams of liquid composition. The liquid may be applied to the substrate via spraying and/or soaking prior to final packaging. The wipe may be wrapped in single use pouches made from foil or plastic or packed in groups of 10, 40, or more in multiple use tubs.

To deliver the benefits of the present invention in this form, the wipe is removed from its package and is rubbed onto the target surface, in a manner that ensures complete wetting of the surface. The wetting practice removes visible dirt and eradicates viruses. Upon drying, the surface experiences anti-viral activity for several hours. Employing this method affords immediate anti-viral action and provides long-lasting activity in regular application.

### Example 10 Anti-bacterial/Anti-Viral All Purpose Cleaner Formulation

| | % BY |
|---|---|
| INGREDIENTS | WEIGHT |
| A. | |
| DEIONIZED WATER | 91.40 |
| NATSURF 265¹⁾ | 1.00 |
| TEXAPON N-70²⁾ | 5.00 |
| B. | |
| KATHON CG³⁾ | 0.10 |
| C. | |
| FORMULA G (ANTIBACTERIAL/ | 2.5 |
| ANTIVIRAL COMPOSITION) | |
| TOTAL | 100 |

¹⁾Natsurf 265, Origin: Croda
²⁾Texapon N-70, Origin: BASF
³⁾Kathon CG’ Origin: DOW

The lotion is produced by mixing all ingredients in Phase A until the solution is clear and adding Kathon CG for preserving. The anti-bacterial/anti-viral all purpose cleaner was prepared by adding Formula G, 2,5% by weight.

This solution may be used to produce an all-purpose cleaner product for topical cleaning on hard surfaces. The mixture is filled in an appropriate PET bottle with a trigger at top. Employing this method affords immediate anti-viral action and provides long-lasting activity in regular application.

### REFERENCES

1. Guo Y-R, Cao Q-D, Hong Z-S, Tan Y-Y, Chen S-D, Jin H-J, Tan K-S, Wang D-Y, Yan Y: The origin, transmission and clinical therapies on coronavirus disease 2019 (COVID-19) outbreak—an update on the status. Military Medical Research 2020, 7(1).

2. Lai C-C, Shih T-P, Ko W-C, Tang H-J, Hsueh P-R: Severe acute respiratory syndrome coronavirus 2 (SARS-CoV-2) and coronavirus disease-2019 (COVID-19): The epidemic and the challenges. International Journal of Antimicrobial Agents 2020, 55(3).

3. Arabi YM, Fowler R, Hayden F G: Critical care management of adults with community-acquired severe respiratory viral infection. Intensive Care Medicine 2020, 46(2):315-328.

4. Lim J, Jeon S, Shin H-Y, Kim M J, Seong Y M, Lee W J, Choe K-W, Kang Y M, Lee B, Park S-J: Case of the Index Patient Who Caused Tertiary Transmission of Coronavirus Disease 2019 in Korea: the Application of Lopinavir/Ritonavir for the Treatment of COVID-19 Pneumonia Monitored by Quantitative RT-PCR. Journal of Korean Medical Science 2020, 35(6).,

5. Wax RS, Christian MD: Practical recommendations for critical care and anesthesiology teams caring for novel coronavirus (2019-nCoV) patients. Canadian Journal of Anesthesia-Journal Canadien D Anesthesie 2020, 67(5):568-576.

6. Rodriguez-Morales A J, MacGregor K, Kanagarajah S, Patel D, Schlagenhauf P: Going global—Travel and the 2019 novel coronavirus. Travel Medicine and Infectious Disease 2020, 33.

7. Wang M, Cao R, Zhang L, Yang X, Liu J, Xu M, Shi Z, Hu Z, Zhong W, Xiao G: Remdesivir and chloroquine effectively inhibit the recently emerged novel coronavirus (2019-nCoV) in vitro. Cell Research 2020, 30(3):269-271.

8. Bo Ram Beck BS, Yoonjung Choi, Sungsoo Park, Keunsoo Kang: Predicting comercially available anti-viral drugs that may act on the novel coronavirus (SARS-CoV-2) through a drug-target interaction deep learning model I Elsevier Enhanced Reader. Computational and Structural Biotechnology Journal 2020, 16:6.

9. Lu H: Drug treatment options for the 2019-new coronavirus (2019-nCoV). Bioscience Trends 2020, 14(1):69-71.

10. Xu Z, Peng C, Shi Y, Zhu Z, Mu K, Wang X, Zhu W: Nel II navir was predicted to be a potential inhibitor of 2019-nCov main protease by an integrative approach combining homology modelling, molecular docking and binding free energy calculation. In.: bioRxiv;

11. Wang Z, Chen X, Lu Y, Chen F, Zhang W: Clinical characteristics and therapeutic procedure for four cases with 2019 novel coronavirus pneumonia receiving combined Chinese and Western medicine treatment. Bioscience Trends 2020, 14(1):64-68.

12. Scientific Committee on Consumer Safety (SCCS) Opinion on Fragrance allergens in cosmetic products. Brussels [https://eur-lex.europa.eu/legal-content/EN/TXT/?uri=CELEX:01976L0768—Page 20/2820120823]

13. Arctander S: Perfume and Flavor Chemicals (Aroma Chemicals), vol. 2. Newark, New Jersey:

Allured Publishing;

14. Muller P M, Lamparsky, D.: Perfumes-Art, Science and Technology. Zurich: Elsevier Applied Science;

15. Berger R G, Pickenhagen W, Kraft P: Scent and Chemistry. The Molecular World of Odors. Zurich:Verlag Helvetica Chimica Acta,;

16. Cerbelaud R: Formulaire De Parfumerie Tome I. Paris: Editions Opera;

17. Arctander S: Perfume and Flavor Materials of Natural Origin, vol. 2. New Jersey: Allured Publishing;

18. Guenther E: The Essential Oils, Vol. 6: Individual Essential Oils. Malabar, Florida: Krieger Pub Co.;

19. Crystal structure of RNA binding domain of nucleocapsid phosphoprotein from SARS coronavirus 2. [https://www.rcsb.org/structure/6VYO]

20. Khupse R, Dixit P: Potential Anti-viral Mechanism of Hydroxychloroquine in COVID-19 and

Further Extrapolation to Celecoxib (Celebrex) for Future Clinical Trials. Authorea 2020.

21. Wiley B. (2015), Wiley Registry 10th Edition/NIST 2014 Mass Spectral Library (Software) CD-ROM ISBN: 978-1-119-00414-1, Scientific Instrument Services, Inc., New Jersey, USA.

22. Wiley B. (2015), Wiley Registry 10th Edition/NIST 2014 Mass Spectral Library Upgrade (Software) CD-ROM ISBN: 978-1-119-00412-7, Scientific Instrument Services, Inc., New Jersey, USA.

23. Wiley B. (2009), Wiley Registry 9th Edition/NIST 2008 Mass Spectrum Library (Software) CD-ROM ISBN-13: 978-1118016718, Scientific Instrument Services, Inc., New Jersey, USA.

24. National Institute of Standards and Technology (US Department of Commerce) (2018), NIST 2017 NIST/EPA/NIH Mass Spectral Library 2017 Edition (Software), Scientific Instrument Services, Inc., New Jersey, USA.

25. Adams, R. P. (2007). Identification of Essential Oil Components by Gas Chromatography/Quadrupole Mass Spectroscopy, 4th ed. Allured Publishing Corp., Carol Stream, Illinois, USA.

26. Adams, R. P. (2007), Essential Oil Identification Mass Spectrum Library (Software), Allured Publishing Corp., Carol Stream, Illinois, USA.

1. An antiviral composition suitable for preventing and/or curing SARS-CoV-2 infection, comprising; one or more essential oils.

2. An antiviral composition according to claim 1, wherein said antibacterial/antiviral composition comprises Formula A comprising:
Benzyl Salicylate
Cinnamon Bark Oil
Curcuma Oil
Eucalyptus Globulus Oil
Tea Tree Oil
Ginger Oil
Rosemary Oil
Benzyl Benzoate.

3. An antiviral composition according to claim 1, wherein said antiviral composition comprises Formula B comprising:
Jatamansi Oil
Gurjun Balsam Oil
Styrax Resinoid
Styrax Oil
Myrrhe Resinoid
Myrrhe Oil
Agarwood Oil
Copaiba Balsam Oil
Ginger Oil
Cypriol Oil
Sandalwood Oil East Indian
Grapefruit Oil
Salvia Sclaree Absolute
Vetiveryl Acetate
L-Muscone
Benzyl Cinnamate
Olibanum Resinoid
Patchouly Oil
Nootkatone
Benzyl Benzoate.

4. An antiviral composition according to claim 1, wherein said antiviral composition comprises Formula C comprising:
Gurjun Balsam Oil
Myrrhe Resinoid
Agarwood Oil
Cypriol Oil
Salvia Sclaree Absolute
Olibanum Resinoid
Benzyl Benzoate.

5. An antiviral composition according to claim 2, wherein said antiviral composition comprises Formula D comprising:
Formula A
Formula C.

6. An antiviral composition according to claim 2, wherein said antiviral composition comprises Formula E comprising:
Formula A
Formula B.

7. An antiviral composition according to claim 2, wherein said antiviral composition comprises Formula F comprising:
Formula A
Formula B
Benzyl Benzoate.

8. An antiviral composition according to claim 1, wherein said antiviral composition is hand sanitizer formulation.

9. An antiviral composition according to claim 1, wherein said cologne formulation comprising deionized water, denaturated alcohol.

10. An antiviral composition according to claim 1, wherein said antiviral composition is shampoo formulation.

11. An antiviral composition according to claim 1, wherein said antiviral composition is electrical diffuser formulation.

12. An antiviral composition according to claim 1, wherein said antiviral composition reed diffuser formulation.

13. An antiviral composition according to claim 1, wherein said antiviral composition is air freshner & sanitizer formulation.

14. An antiviral composition according to claim 1, wherein said antiviral composition is air nebulizer formulation.

15. An antiviral composition according to claim 1, wherein said antiviral composition is hand cream formulation.

16. An antiviral composition according to claim 1, wherein said antiviral composition is wet wipe formulation.

17. An antiviral composition according to claim 1, wherein said antiviral composition is cleaner formulation.
